(19) United States
(12) Patent Application Publication
(10) Pub. No.: US 2026/0345989 A1
(43) Pub. Date: Nov. 5, 2026

(54) EXPANDABLE FLOW DETECTION SYSTEM FOR ALLEVIATING LYMPHATIC SYSTEM CONGESTION

(71) Applicant: The Children's Hospital of Philadelphia, Philadelphia, PA (US)

(72) Inventors: Yoav Dori, Philadelphia, PA (US); Denise Merrill, Philadelphia, PA (US); Digvijay Shinde, Philadelphia, PA (US)

(21) Appl. No.: 19/101,955

(22) PCT Filed: Aug. 7, 2023
(86) PCT No.: PCT/US2023/029643

§ 371 Date: Feb. 7, 2025

Related U.S. Application Data
(60) Provisional application No. 63/395,956, filed on Aug. 8, 2022.

(51) Int. Cl. A61M 60/109; A61M 27/00; A61M 60/279; A61M 60/36

(52) U.S. Cl. CPC A61M 60/109; A61M 60/279; A61M 60/36; A61M 2027/004; A61M 2202/0405

(57) ABSTRACT

An expandable flow detector (EFD) is configured for use in systems and methods for decompressing the lymphatic system. The EFD is used to manually, semi-automatically, or automatically control a lymph pump that pumps lymph fluid from the lymphatic system into the venous system and/or out of the body. An expandable fluid reservoir stores lymph fluid for extraction or recirculation into the lymphatic system. A control system and method control the operation of the lymph pump to control thoracic duct pressure and flow of lymph during decompression. Desirable conditions in the EFD may be created by adjusting the height difference between the EFD and the subject and/or adjusting the pressure around the EFD. The resultant system may be used for inpatient and outpatient applications, for continuous or intermittent use, and may be inserted into the subject's thoracic duct, inserted subcutaneously, or used externally (e.g., as a wearable).

### PRIORITY CLAIM

The present patent application claims priority to U.S. Provisional Patent Application No. 63/395,956, filed Aug. 8, 2022, the content of which is hereby incorporated by reference in its entirety and for all purposes.

### TECHNICAL FIELD

This disclosure describes an expandable flow detector and reservoir and, more generally, a system that uses the expandable flow detector and reservoir to alleviate congestion of the lymphatic system.

### BACKGROUND

According to the National Heart, Lung and Blood Institute (NHLBI), there are approximately 5 million Americans living with congestive heart failure (CHF) and the prevalence of liver cirrhosis is estimated at 400,000 individuals. Both diseases are associated with significant morbidity and mortality. Abnormalities in lymphatic production and drainage are responsible for many symptoms in these diseases. In CHF, increased central venous pressure can lead to decreased lymphatic drainage and increased lymphatic production, which results in CHF symptoms such as tissue edema and liver enlargement. In subjects with liver cirrhosis, increased pressure in the hepatic sinusoid can lead to significant increases in liver lymphatic production. When production overcomes the capacity of the lymphatic system to drain, symptoms can appear in the form of ascites, an excess of fluid in the peritoneal cavity.

Most of the lymphatic fluid that originates in the tissue drains into the thoracic duct (TD), which is the largest lymphatic vessel in the body. It has been shown that the TD can be significantly distended in subjects with CHF and liver cirrhosis. The cause of TD distension is likely due to an increased production of lymph fluid and elevated TD pressure. It has been shown that in subjects with CHF, external decompression of the TD can improve symptoms of CHF, such as dyspnea, orthopnea, anorexia, abdominal discomfort, distended neck veins, hepatomegaly, peripheral and scrotal edema and ascites. Decompression of the TD has also been shown to resolve ascites in subjects with liver cirrhosis.

External drainage of the TD is invasive and can lead to significant metabolic, immunologic, and fluid imbalances. Internal drainage of the TD can overcome these limitations and has been demonstrated to work in both animals and humans. For example, TD to subclavian vein anastomosis to resolve cirrhotic ascites has been described by Akcay, et al. in “Effects of operative manipulation on the flow of intestinal lymphatics,” Am. J. Surg., 1971 November; 122(5): 662-5, and by Schreiber, et al. in “Cervical lymphovenous anastomosis for portal hypertension in cirrhosis of the liver,” Ger Med Mon., 1968 August; 13(8): 361-6. A TD to pulmonary vein shunt has also been described but can require a complicated surgical procedure and its long-term efficacy is unknown. One of the problems with passive decompression of the TD into the venous system is that in most subjects with CHF the pressure in the central venous system is elevated and this increase in pressure has been shown to impede the flow from the TD into the venous system.

A system and method for providing long-term decompression of the lymphatic system and to alleviate the symptoms associated with CHF and liver cirrhosis has been described in U.S. Pat. No. 10,639,458. The system described in U.S. Pat. No. 10,639,458 includes a pump and a controller coupled to the pump. A sensor coupled to the controller detects changes in pressure in the thoracic duct and/or the central venous system and, when an increase in pressure is detected, the controller activates the pump to remove lymph fluid from the lymphatic system. A pressure controlled chamber also provides a favorable pressure gradient for the flow of lymph fluid from the lymphatic system into the device, and the pressure controlled chamber can be coupled to the pump and/or the controller. A device such as a stent and a valve coupled to the stent may be delivered transcutaneously directly into the TD and implanted into the junction between the venous system and the TD and connected to the pump and controller. Components of the device also can be positioned on top of the skin or under the skin of the subject. The system including such a device passively reduces the congestion of the lymphatic system.

It is desired to provide a system and method for providing long-term decompression of the lymphatic system that uses an external pump and that may be used in inpatient as well as outpatient settings for continuous or intermittent use.

### SUMMARY

Various examples are now described to introduce a selection of concepts in a simplified form that are further described below in the Detailed Description. The Summary is not intended to identify key or essential features of the claimed subject matter, nor is it intended to be used to limit the scope of the claimed subject matter.

The present disclosure relates to examples of devices for decompressing the lymphatic system as well as methods for deploying and using such devices. For example, an expandable flow detector and reservoir adapted to measure flow of lymphatic fluid are provided in a device for reducing congestion of a lymphatic system. The expandable flow detector and reservoir includes an inlet tubular member that receives lymphatic fluid from the lymphatic system, an expandable reservoir that expands under pressure and/or flow and deflates when the pressure and/or flow is reduced, a sealed housing around the expandable reservoir including a fluid that is compressed as the reservoir expands, an outlet tubular member that provides the lymphatic fluid to a pump, and a sensor that measures expansion or contraction of at least one of the lymphatic fluid within the reservoir or compression or expansion of the fluid in the sealed housing as the reservoir expands or contracts. In sample configurations, the expandable reservoir includes at least one connector or valve that receives the inlet tubular member and at least one connector or valve that receives the outlet tubular member.

In sample configurations, the expandable flow detector and reservoir further includes a movable element that is displaced as the reservoir expands whereby the displacement is indicative of a change in pressure and/or flow in at least one of the reservoir or the housing. For example, the expandable reservoir may be formed by the housing and a movable piston that is displaced as lymphatic fluid is received from the lymphatic system whereby the displacement is indicative of a change in the amount of received lymphatic fluid. In other configurations, the housing may include a movable housing element that is movable in response to pressure and/or flow as a result of the volume of fluid within the housing expanding as the expandable reservoir is filled with lymphatic fluid.

In other sample configurations, a fluid chamber is provided that communicates with the fluid in the housing. A sensor may also be provided that is adapted to measure changes in volume of the fluid in the fluid chamber as a result of changes in pressure and/or flow of the lymphatic fluid received by the expandable reservoir as the expandable reservoir expands or contracts during use. A support may also be provided for the fluid chamber. In sample configurations, the fluid chamber is connected to the support in an adjustable manner whereby a height of the fluid chamber relative to the housing may be adjusted. An expandable fluid reservoir may also be provided to capture fluid overflow based on a relative level of the expandable fluid reservoir to the fluid chamber.

The description herein further relates to a device for reducing congestion of a lymphatic system using an expandable flow detector and reservoir. The device is connected to a first catheter or cannula adapted for insertion into a thoracic duct of a subject to extract lymphatic fluid from the thoracic duct of the subject and to a second catheter or cannula that receives the lymphatic fluid from a pump (e.g., peristaltic pump) and that is adapted for insertion into a vein of a subject to insert lymphatic fluid into the subject. The device may include an inlet tubular member that receives lymphatic fluid from the first catheter or cannula, an expandable reservoir that receives the lymphatic fluid from the inlet tubular member and expands under pressure and/or increased flow and deflates when the pressure and/or the flow is reduced, a sealed housing around the expandable reservoir including a fluid that is compressed as the expandable reservoir expands, a sensor that measures expansion or contraction of at least one of the lymphatic fluid within the expandable reservoir or compression or expansion of the fluid in the sealed housing as the expandable reservoir expands or contracts, a pump, and an outlet tubular member that outputs the lymphatic fluid from the expandable reservoir to the pump. The device may further include an expandable or fixed fluid reservoir that is adapted to receive excess lymphatic fluid. The expandable or fixed fluid reservoir may be connected between the expandable reservoir and the pump, directly to the expandable reservoir within the housing, or connected after the pump.

In other configurations, the device further includes a signal processor and filter that receives a signal from the sensor and determines a variability in a fluid flow rate through the expandable reservoir and filters out noise signals. The device may also include a controller that receives a processing output from the signal processor that it uses to control the pump. The controller may further control at least one of activation, deactivation or pressure level of at least one of the expandable reservoir or the pump. The controller may be positioned remotely from the pump so as to communicate with the pump through at least one of a wired or wireless connection. An input device also may be provided that provides a set point to the controller for controlling the pump and sensor to maintain desired thoracic duct outlet and interstitial pressures.

The pump may be controlled automatically or semi-automatically. For example, the pump may be controlled to automatically increase pump flow when the sensor detects increased lymphatic fluid flow or pressure and to automatically decrease pump flow when the sensor detects decreased lymphatic fluid flow or pressure. A plurality or pressure sensors may be placed at one or more locations including a proximal end of the first catheter or cannula, a proximal end of the second catheter or cannula, at an entrance to the pump, and an exit of the pump. The location of an occlusion in the device may be detected by detecting pressure changes between respective pressure sensors. Upon detection of an occlusion, the pump may be shut off until the occlusion has been cleared.

In one sample configuration, the device may include a second pump that pumps excess lymphatic fluid output by the pump to an expandable or fixed fluid reservoir.

In other sample configurations, the expandable reservoir, sealed housing, sensor, and pump may have sizes adapted to enable the device to be insertable into a thoracic duct, a subclavian vein, or other veins of a subject, inserted subcutaneously, or provided externally (e.g., as a wearable).

A method of reducing congestion of a lymphatic system is also described using a device having an expandable flow detector and reservoir. The method includes inserting a first catheter or cannula into a thoracic duct of a subject in a position to extract lymphatic fluid from the thoracic duct of the subject and inserting a second catheter or cannula into a vein of the subject in a position to insert lymphatic fluid into the vein of the subject. During continuous or intermittent use, a device is connected between the first catheter or cannula and the second catheter or cannula. The device includes an inlet tubular member that receives lymphatic fluid from the first catheter or cannula, an expandable reservoir that receives the lymphatic fluid from the inlet tubular member and expands under pressure and/or flow and deflates when the pressure and/or flow is reduced, a sealed housing around the expandable reservoir including a fluid that is compressed as the expandable reservoir expands, a sensor that measures expansion or contraction of at least one of the lymphatic fluid within the expandable reservoir or compression or expansion of the fluid in the sealed housing as the expandable reservoir expands or contracts, a pump, and an outlet tubular member that outputs the lymphatic fluid from the expandable reservoir to the pump. The output of the pump is connected to the second catheter or cannula to provide lymphatic fluid output by the pump. The device may be delivered transcutaneously directly into the thoracic duct of the subject or at least one component of the device may sit on top of skin of the subject. The device also may be surgically attached to the thoracic duct of the subject or implanted subcutaneously under skin of the subject. In any of these configurations, operation of at least one component of the device may be controlled using a remote control device.

The explanations provided for each of the above-mentioned aspects and its implementation apply equally to the other aspects and the corresponding implementations. The processing configurations may be implemented in hardware, software, or any combination thereof. Also, any one of the foregoing examples may be combined with any one or more of the other foregoing examples to create a new configuration within the scope of the present disclosure.

### DETAILED DESCRIPTION OF THE DRAWINGS

The foregoing and other beneficial features and advantages of the subject matter described herein will become apparent from the following detailed description in connection with the attached figures, of which:

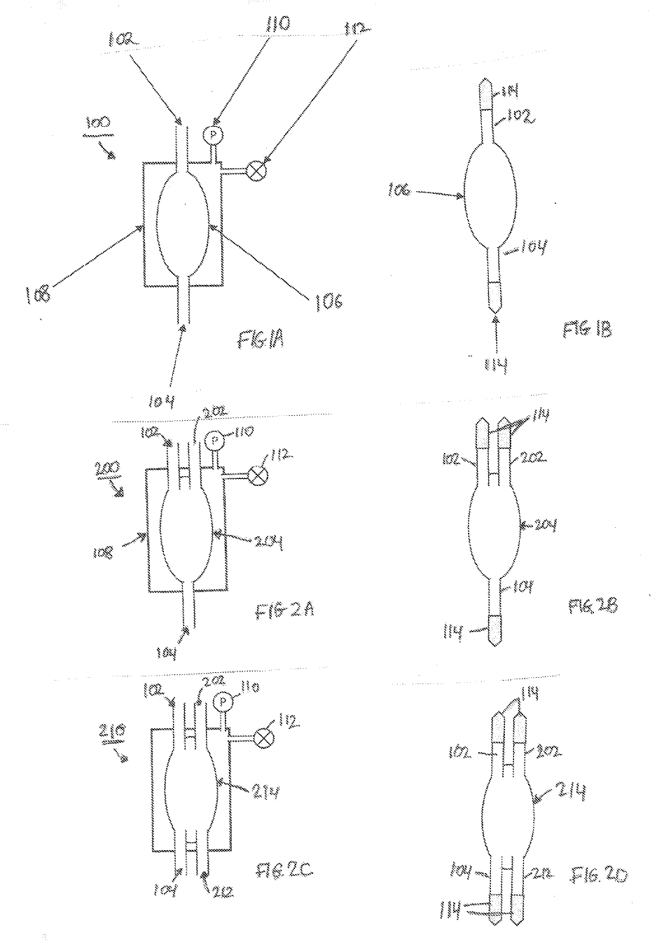

FIG. 1A illustrates an example of an expandable flow detector (EFD) and reservoir in a sample configuration.

FIG. 1B illustrates a sample expandable reservoir for the EFD and for use as an excess reservoir in the sample configuration of FIG. 1A.

FIG. 2A illustrates another example of an EFD and reservoir having two inlet ports in a sample configuration.

FIG. 2B illustrates a sample expandable reservoir for the EFD and for use as an excess reservoir in the sample configuration of FIG. 2A.

FIG. 2C illustrates another example of an EFD and reservoir having two inlet ports and two outlet ports in a sample configuration.

FIG. 2D illustrates a sample expandable reservoir for the EFD and for use as an excess reservoir in the sample configuration of FIG. 2C.

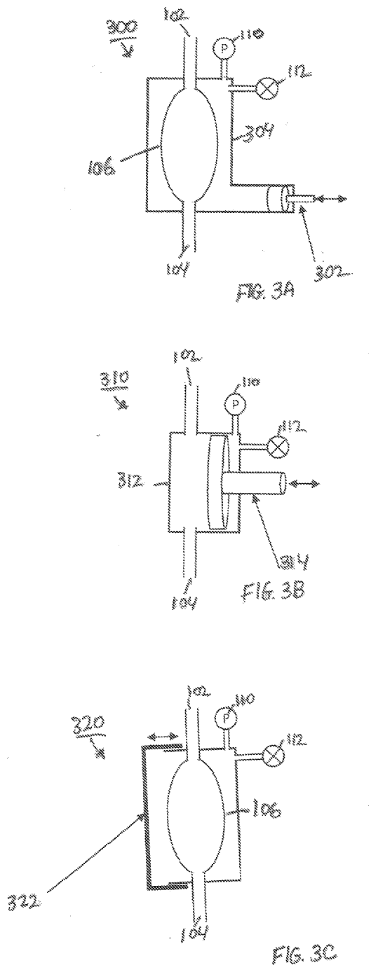

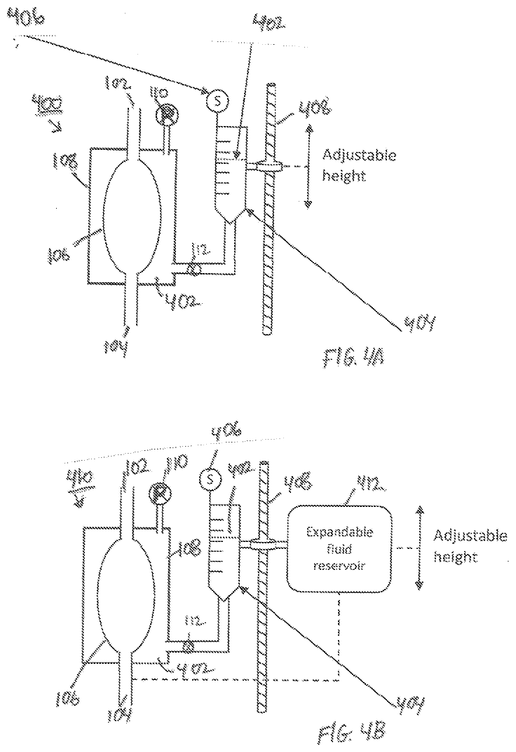

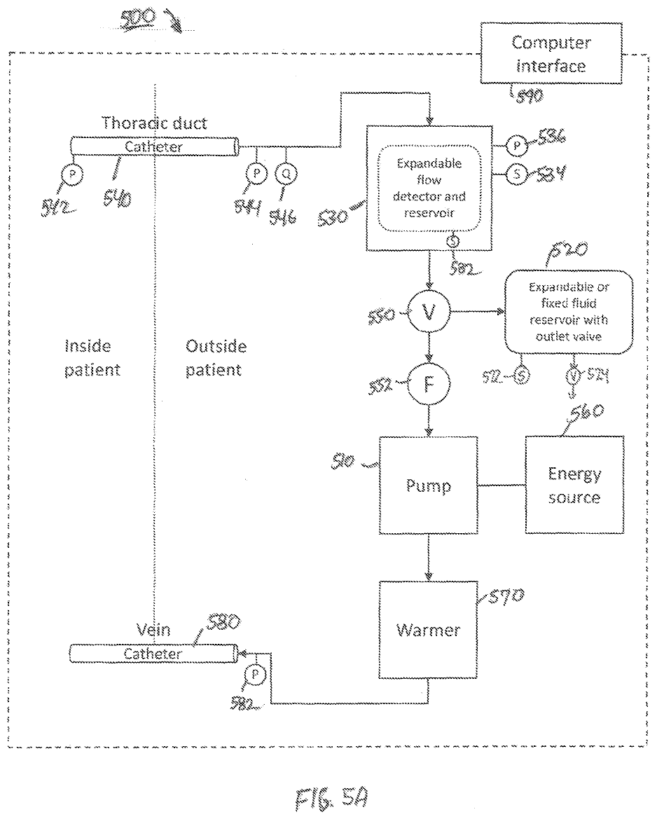

FIG. 3A illustrates an example of an EFD and reservoir including a movable piston in a sample configuration.

FIG. 3B illustrates another example of an EFD and reservoir including a movable piston in a sample configuration.

FIG. 3C illustrates another example of an EFD and reservoir including a movable housing element in a sample configuration.

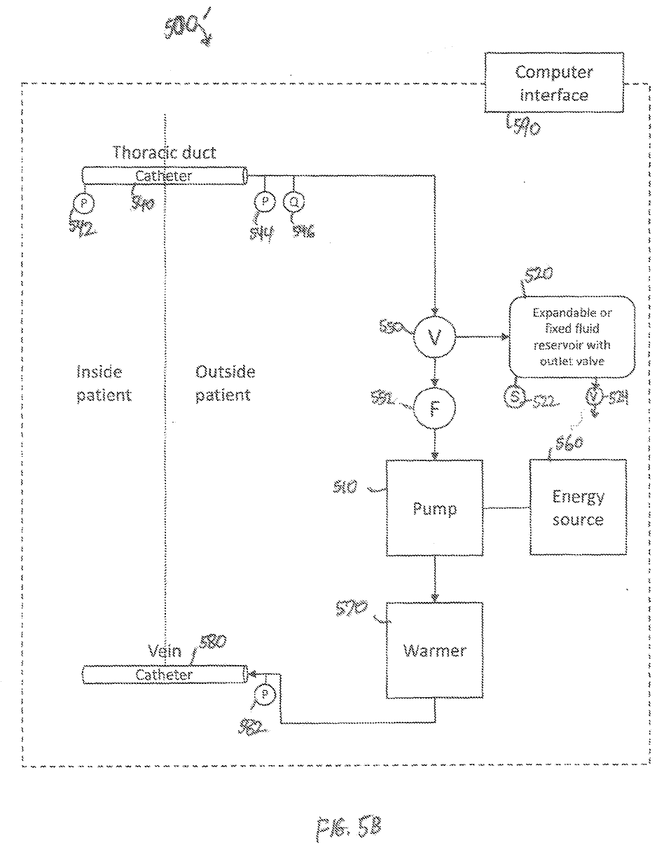

FIG. 4A illustrates an example of an EFD and reservoir surrounded by a fluid such as water, where the water volume may be used to determine the volume of lymphatic fluid stored in the reservoir.

FIG. 4B illustrates another example of an EFD and reservoir surrounded by a fluid such as water, where the water volume may be used to determine the volume of lymphatic fluid stored in the reservoir.

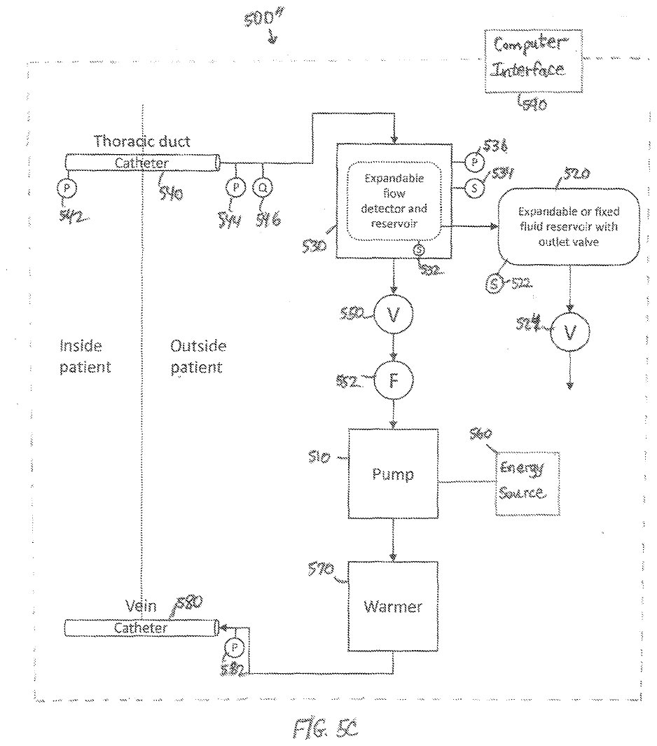

FIG. 5A illustrates an example of a system for manual or semi-automatic operation of a lymphatic pump where the excess fluid reservoir is located post the EFD and reservoir in a sample configuration.

FIG. 5B illustrates an example of a system for manual or semi-automatic operation of a lymphatic pump where the only fluid reservoir is used as the EFD in a sample configuration.

FIG. 5C illustrates an example of a system for manual or semi-automatic operation of a lymphatic pump where the excess fluid reservoir is connected directly to the reservoir of the EFD in a sample configuration.

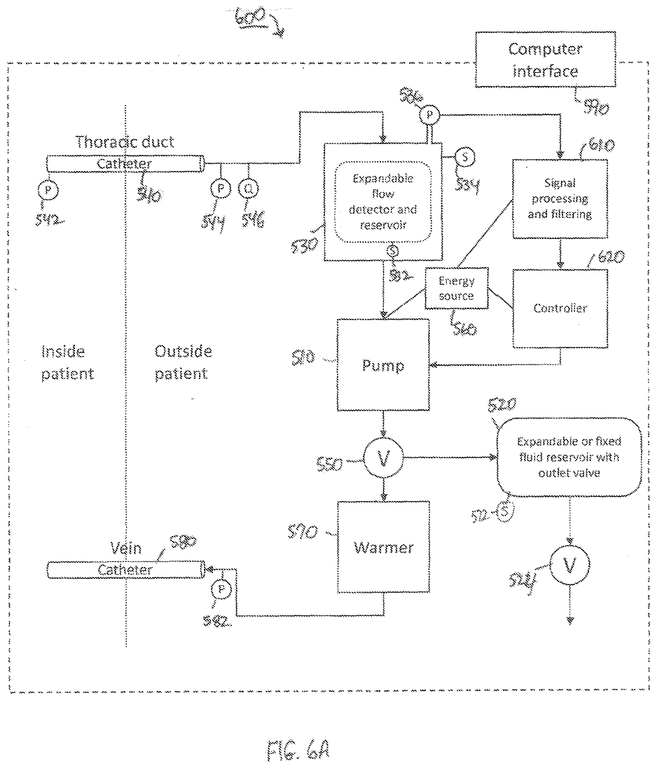

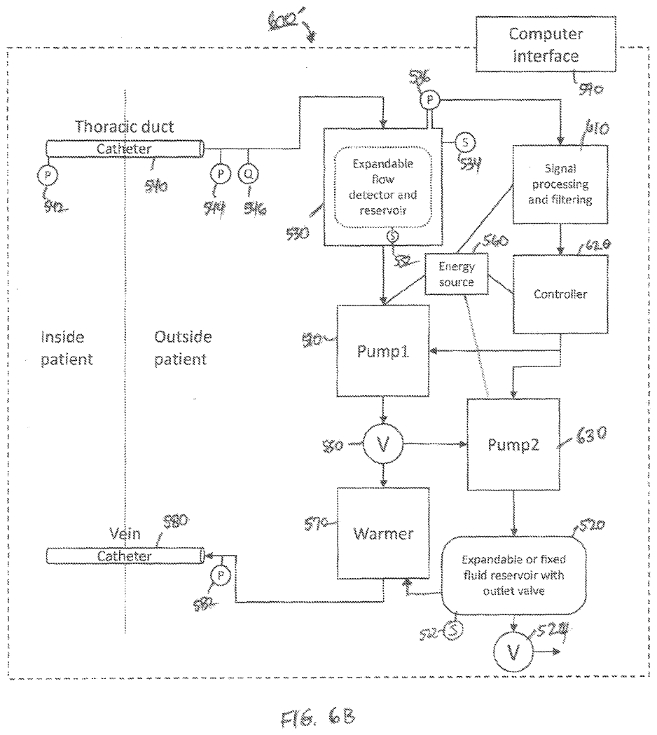

FIG. 6A illustrates an example of a system for automatic operation of a lymphatic pump where the excess reservoir or outlet valve is located post the pump in a sample configuration.

FIG. 6B illustrates an example of a system for automatic operation of a lymphatic pump where the excess reservoir or outlet valve is located post the pump and a second lymphatic pump is provided for a partial lymphatic fluid return operation in a sample configuration.

FIG. 6C illustrates an example of a system for automatic operation of a lymphatic pump where the excess reservoir or outlet valve is located before the pump in a sample configuration.

FIG. 7A illustrates an example of a system for automatic operation of a small wearable or implantable device including an EFD and reservoir in a sample configuration.

FIG. 7B is a plot illustrating the response of a pump to a drop in the EFD pressure set point in a sample configuration.

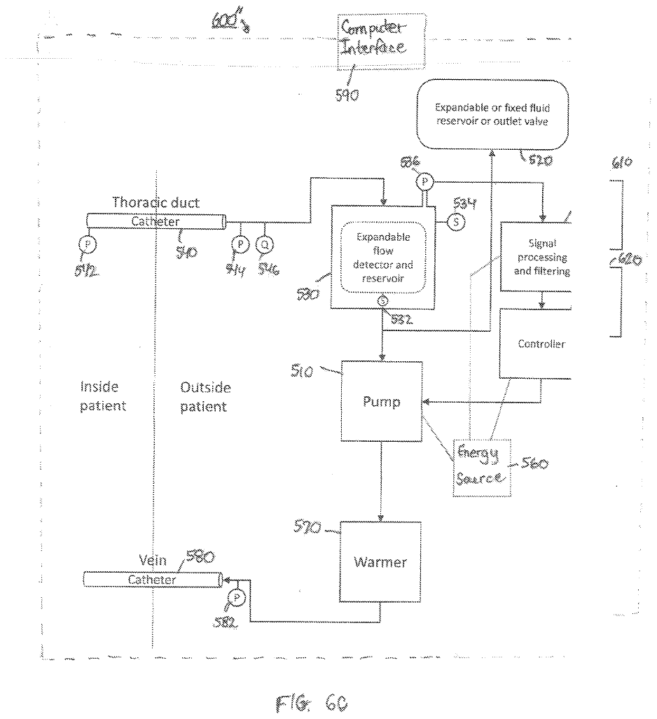

FIGS. 8A and 8B illustrate sample configurations of peristaltic pumps for use as the lymphatic pump in sample system configurations.

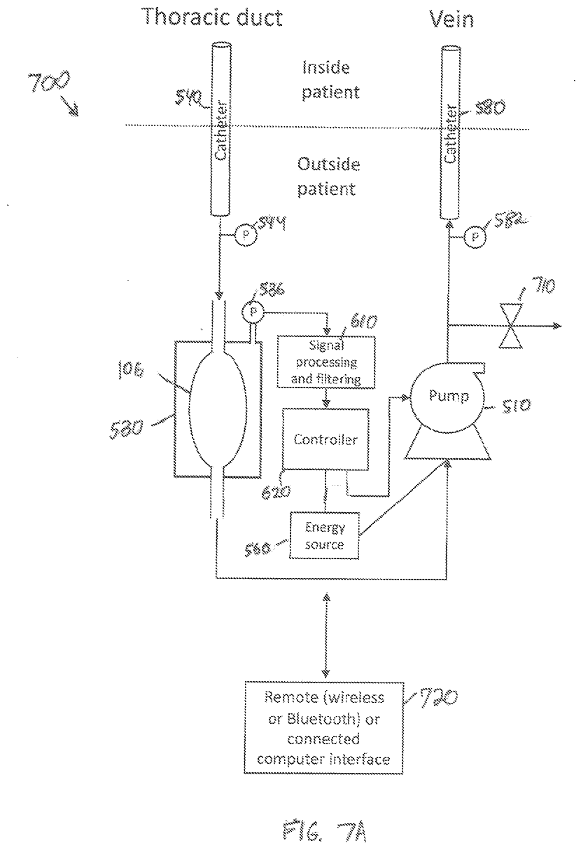

FIG. 9 is a diagram showing the variable placement of the EFD and reservoir in relation to the subject's position.

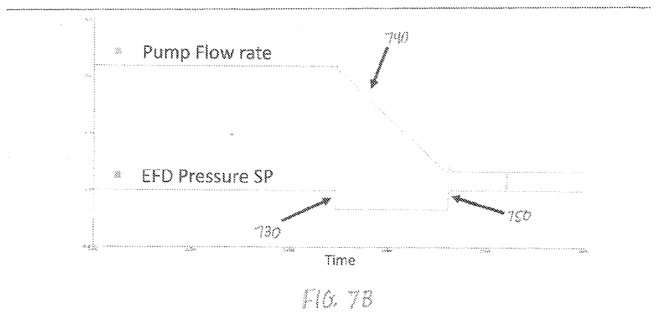

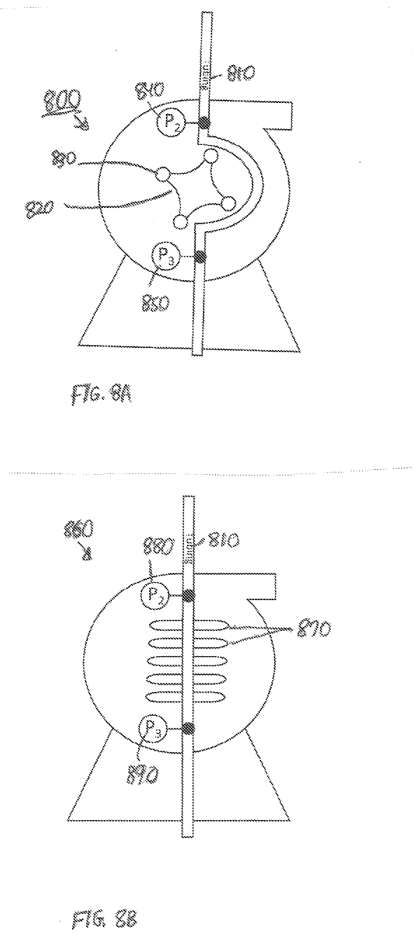

FIG. 10 is a diagram showing the possibility of the EFD and reservoir having a modifiable relative height in a sample configuration.

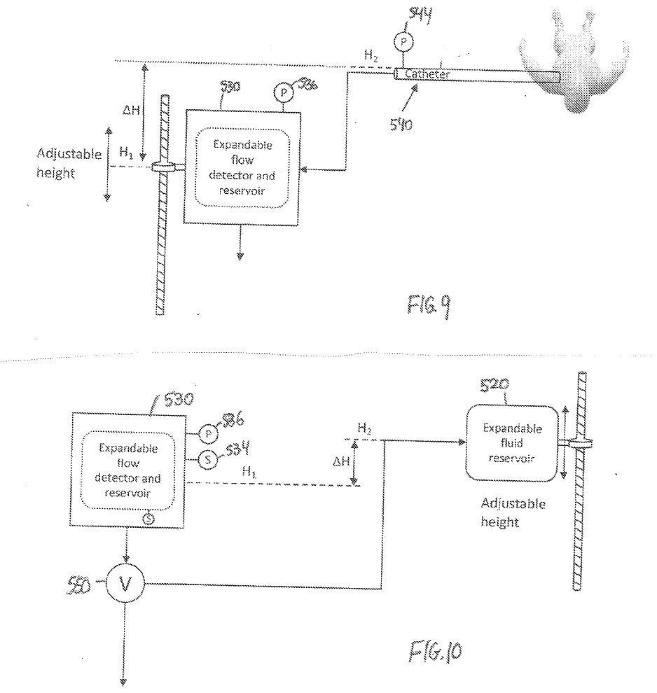

FIG. 11A is a diagram showing possible catheter and cannula positions and a TD entrance in a sample configuration.

FIG. 11B is a diagram showing a transcervical approach for inserting the catheter and cannula into the TD in a sample configuration.

FIG. 11C is a diagram showing a venous approach for inserting the catheter and cannula into the TD in a sample configuration.

FIG. 11D is a diagram showing a surgical approach to inserting the catheter and cannula into the TD in a sample configuration.

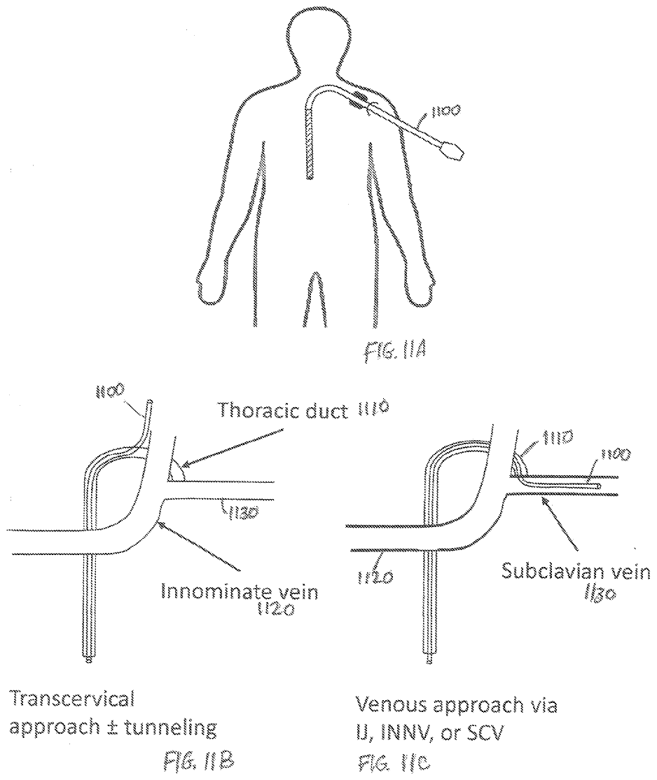

FIG. 12 illustrates a TD cannula and catheter having a portion of the catheter braded to allow bending but preventing compression in a sample configuration.

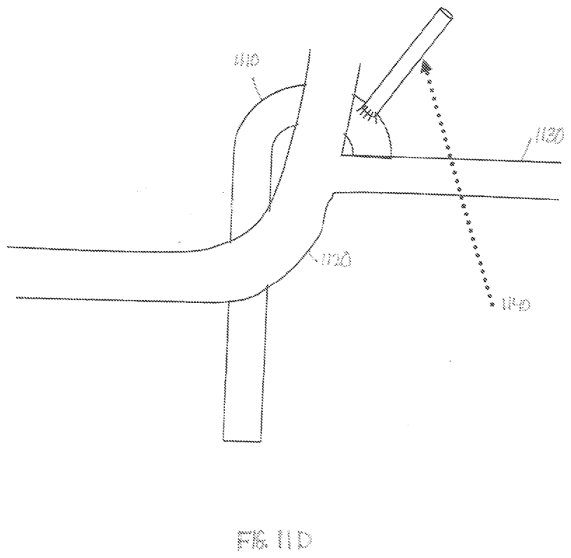

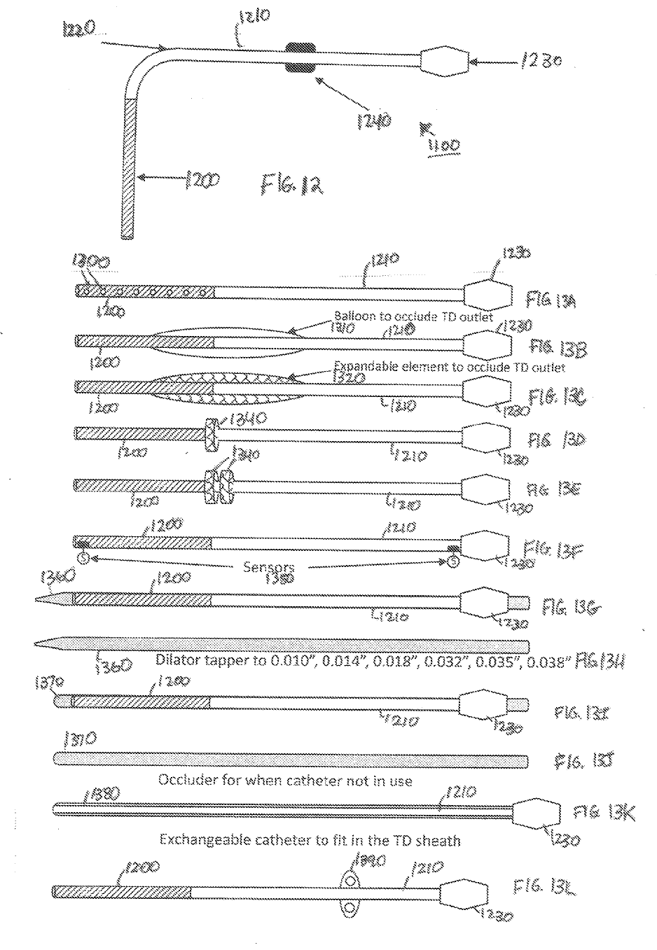

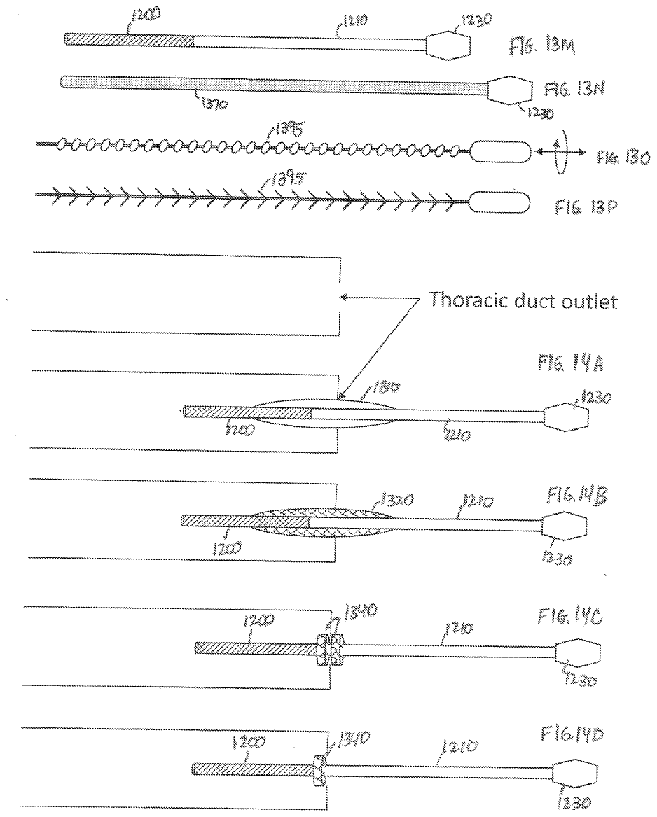

FIGS. 13A-13P illustrate respective configurations of a TD cannula and catheter adapted for inserting into the TD.

FIGS. 14A-14D illustrate insertion of a TD cannula and catheter into the TD and different mechanisms for holding the TD cannula and catheter in place in sample configurations.

FIGS. 15A-15C illustrate respective catheter configurations for providing side access into the TD in sample configurations.

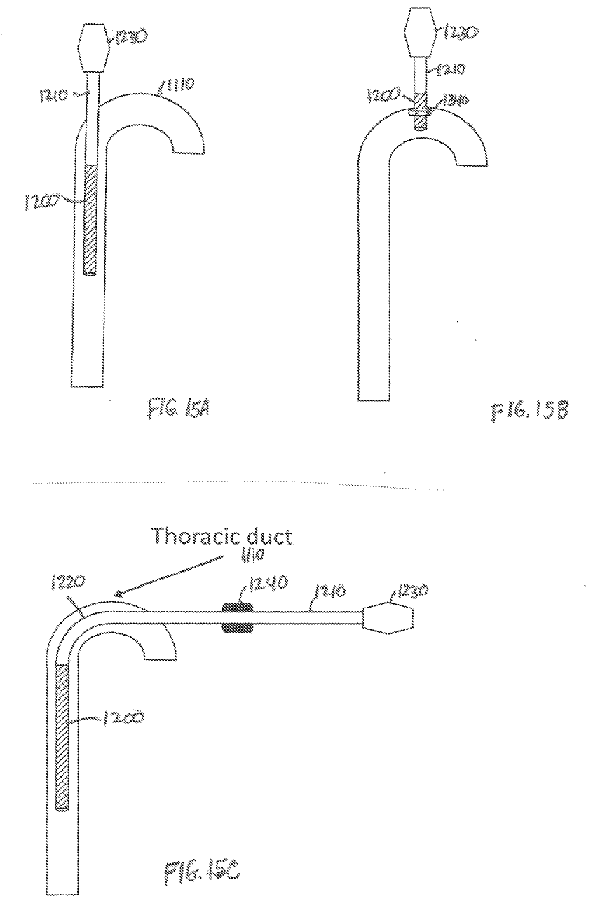

FIG. 16 illustrates the modes of operation of the system in a sample configuration.

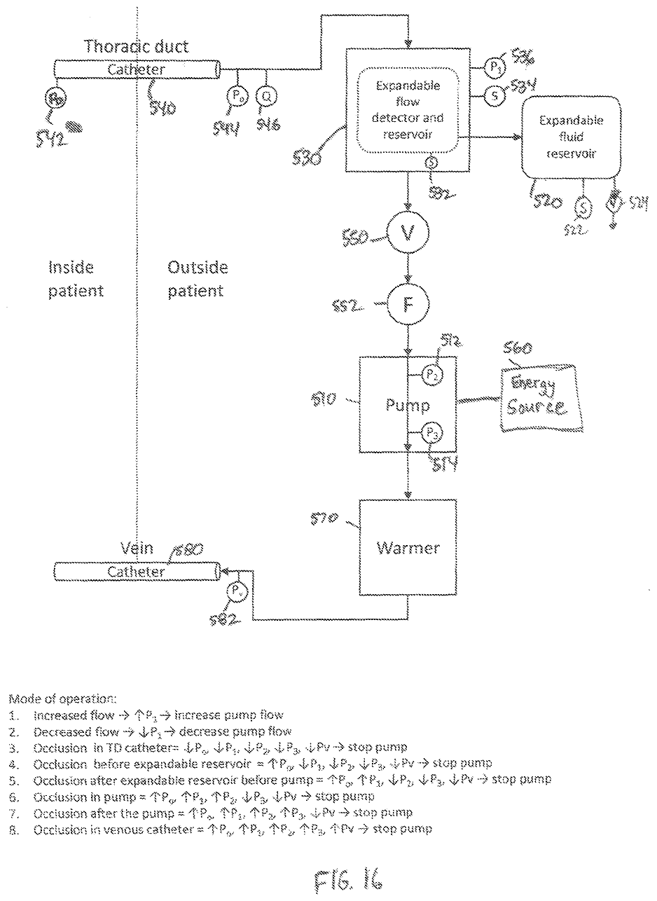

FIG. 17 illustrates a system for providing TD outlet pressure control with an automatic pump in a sample configuration.

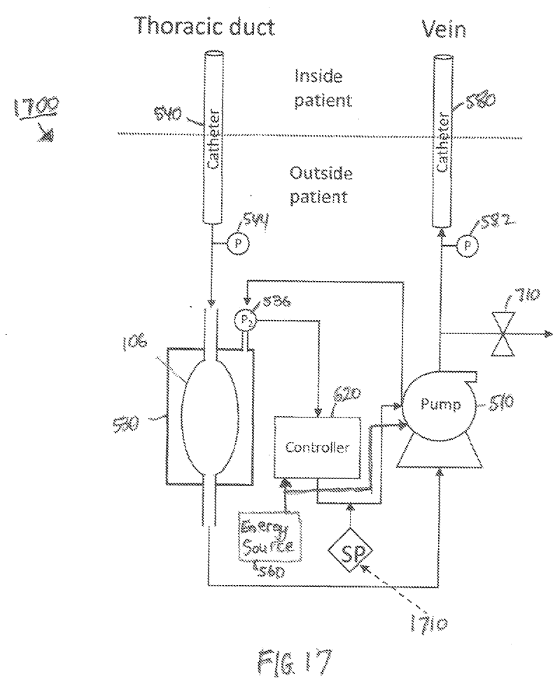

FIG. 18 is a block diagram illustrating circuitry for performing methods and implementing processing features according to example configurations.

### DETAILED DESCRIPTION OF ILLUSTRATIVE CONFIGURATIONS

The following description relates to methods and devices for decompressing the lymphatic system. For example, the following description describes with respect to FIGS. 1-18 devices for actively or passively decompressing the lymphatic system and methods for the deployment and use of such devices within a subject.

As described in U.S. Pat. No. 10,639,458, one of the functions of the lymphatic system is to remove the excess interstitial fluid from body tissues and to deliver the interstitial fluid to the venous system. Fluid perfusion of the tissues involves the filtration of the fluid portion of the blood (i.e., plasma) through the arterial capillary network into interstitial tissue resulting in an accumulation of interstitial fluid. A large portion of the fluid within the tissues is returned (e.g., approximately 80%) to the venous system. The remaining fluid (i.e., the lymph) can be removed from the tissues by the lymphatic system (e.g., approximately 20%). The lymphatic system mat return this excess fluid into the venous system through a valve at the junction of the thoracic duct and the innominate vein.

Lymph production is governed by the Starling equation set forth in Equation 1:

Jv=Kf([PC−Pi)−σ[πC−πi]) Equation 1

where Jv is the net fluid movement between compartments; PC is the capillary hydrostatic pressure; Pi is the interstitial hydrostatic pressure; πC is the capillary oncotic pressure; πi is the interstitial oncotic pressure; Kf is the filtration coefficient (a proportionality constant); and σ is the reflection coefficient.

Lymphatic flow from tissue into the thoracic duct (TD) and then out of the TD outlet into the venous system is governed by the dynamic pressure gradients between the tissue and the TD and between the TD and the venous system as well as by the cross-sectional area available for fluid flow. This relationship can be described by the Hagen-Poiseuille equation set forth in Equation 2:

$$Q = \frac{Δ P \cdot π {r}^{4}}{8 μ L} \qquad Equation 2$$

where Q is the volumetric flow rate, ΔP is the pressure gradient between the two ends of the vessel or duct; r is the radius of the vessel or opening (e.g., duct), μ is the dynamic viscosity, and L is the length of the vessel or duct.

In the absence of proper drainage of the lymphatic system into the venous system, congestion of the lymphatic system and central lymphatic failure can result. Central lymphatic failure can occur under circumstances where lymph production exceeds the lymphatic drainage capacity of the lymphatic system. This can occur in the setting of CHF where there is increased lymphatic production due to a shift in the Starling forces as well as inhibition of the lymphatic drainage due to an elevated central venous pressure and a limited flow capacity of the TD outlet valve. In subjects with liver cirrhosis, increased production of lymph in the liver can exceed the flow capacity of the TD outlet valve in the setting of normal central venous pressure resulting in central lymphatic failure.

Congestion of the lymphatic system leads to the accumulation of a watery fluid in the tissue causing tissue swelling and edema. Tissue edema in the lungs can lead to orthopnea and dyspnea, and tissue edema in the liver can lead to liver enlargement and dysfunction. Tissue edema can also interfere with wound healing and, if left untreated, can cause fibrosis. Fibrosis, which is a hardening of the tissue in the affected area, can further complicate the drainage process and can cause life-threatening conditions, such as infections.

The following description relates to devices and methods for alleviating the symptoms associated with lymphatic diseases and in conditions where drainage of the lymphatic system is needed such as, but not limited to, CHF, congenital heart disease, right-sided heart failure, Noonan syndrome, Turner syndrome, liver cirrhosis, lymphatic disease such as Gorham's disease, central conducting lymphatic anomaly (CCLA), kaposiform lymphangiomatosis (KLA), generalized lymphatic anomaly (GLA), lymphangioleiomyomatosis, lymphangiectasia, multiple sclerosis, human immunodeficiency virus (HIV), autoimmune diseases, rheumatologic diseases such as rheumatoid arthritis, infectious diseases other than HIV, systemic inflammatory response syndrome (SIRS), acute respiratory distress syndrome (ARDS), lung disease, bronchopulmonary dysplasia (BPD), pulmonary hypertension, immune deficiencies, cancer and during cancer therapy. The following description also relates to methods for implanting and using such devices.

Unless otherwise defined, all technical and scientific terms used herein have the same meanings as commonly understood by one of ordinary skill in the art.

An “individual,” “patient,” or “subject,” as used interchangeably herein, can be a human or non-human animal. Non-limiting examples of non-human animal subjects including non-human primates, dogs, cats, mice, rats, guinea pigs, rabbits, pigs, fowl, horses, cows, goats, sheep, and cetaceans.

The term “coupled,” as used herein, refers to the connection of a device component to another device component by any means known in the art. The type of coupling used to connect two or more device components can depend on the scale and operability of the device. For example, and not by way of limitation, coupling of two or more components of a device disclosed herein can include one or more joints, valves, fittings, couplings, transfer lines, or sealing elements.

In the configurations described herein, the devices for decompressing the lymphatic system to alleviate the symptoms associated with central lymphatic failure include an expandable flow detector (EFD) that works within the disclosed system to either actively or passively reduce the congestion of the lymphatic system by alleviating unfavorable pressure gradients and/or the limited flow capacity of the outlet valve. The EFD is used to manually, semi-automatically, or automatically control a lymph pump that pumps lymphatic fluid from the lymphatic system. An expandable fluid reservoir is also provided for storing lymph for extraction or recirculation into the lymphatic system. A control system and method control the operation of the lymph pump to control thoracic duct pressure and flow of lymph during decompression. Desirable conditions in the EFD may be created by adjusting the height difference between the EFD and the subject and/or adjusting the pressure around the EFD. The resultant system may be used for inpatient and outpatient applications, for continuous or intermittent use, and may be inserted into the subject's thoracic duct or vein surgically or transcutaneously, inserted subcutaneously or into a compartment of the body, or used externally (e.g., as a wearable).

The system includes a pump that decompresses the thoracic duct by pumping lymphatic fluid out of the thoracic duct. The pump may be implanted into the thoracic duct or subclavian or other vein, may be inserted subcutaneously or into a compartment of the body, may be included in a wearable, or may be an external pump that is not part of a wearable. The EFD may be adapted to include a collection chamber that acts as both a flow detector and reservoir. In operation, the lymph drains into the EFD manually, semi-automatically, or automatically. In manual mode, the user periodically measures the amount of lymph in the EFD and reservoir, records time, and adjusts the pump accordingly. In the semi-automatic mode, sensors measure changes in the EFD and translates the changes to a change in flow into the EFD and then provides the user with pump adjustment guidance. The user manually adjusts the pump. On the other hand, in the automatic mode, sensors measure changes in the EFD and reservoir and continuously adjust the pump. A wearable version reduces the size of the EFD and pump and removes the filter and warmer. An electronic filtering system may filter the EFD sensor signal to allow for smoother operation.

The system described herein thus creates conditions around the EFD to control the thoracic duct outlet pressure and the flow of lymph. To create desirable conditions in the EFD, a height difference between the EFD and the subject may be adjusted. Also, the pressure around the EFD may also be adjusted. In addition to the EFD, the expandable fluid reservoir may act to remove a portion (0%-100%) of the lymph from the lymphatic system. The fluid reservoir may be located externally, and some of the fluid from the reservoir may be discarded. Several different configurations including such features and additional features are described below.

FIG. 1A illustrates an example of an expandable flow detector and reservoir 100 in a sample configuration. As illustrated, the expandable flow detector and reservoir 100 includes an inlet tube 102, an outlet tube 104, and an expandable reservoir 106 made of polyvinyl chloride (PVC), silicone, nylon, rubber, or another suitable expandable material that may expand under pressure and deflate when the pressure is removed. In the configuration of FIG. 1A, the expandable reservoir 106 is disposed within a rigid sealed housing 108 that may include a fluid (e.g., water or air) between the housing 108 and the expandable reservoir 106. As the reservoir 106 fills with fluid, the reservoir 106 expands and causes fluid to press against the housing 108. The pressure or weight of the fluid that causes the expansion may be measured to determine the flow of lymphatic fluid and used in the system to balance the pressures between the venous and the lymphatic systems. It will be appreciated that a small reservoir 106 may be desired for continuous flow, while a larger reservoir 106 may be used for intermittent flow. It will be appreciated that the design prevents the lymphatic fluid from contacting the air.

A pressure sensor 110 may be used to track changes in the pressure of the fluid in the housing 108 outside the expandable reservoir 106 as the expandable reservoir expands or contracts. An access port or valve pressure inlet 112 may also be provided as an outlet for volume measurements (e.g., see FIGS. 4A and 4B) or as an outlet to relieve the pressure due to excess lymphatic fluid in the expandable reservoir 106.

In certain configurations, the valves can be of any type known in the art. For example, but not by way of limitation, the access port or valve 112 can have the configuration of a ball valve, a bicuspid valve, a slit-like valve or a leaflet (e.g., tri-leaflet) valve. Additional non-limiting examples of valves include membrane type, solenoid, mechanical and biological valves. The position of the valve or valves can be anywhere in a stent, a sheath, a tubular element, a vein or in the TD. In certain configurations, valves can be placed in the thoracic duct outlet.

FIG. 1B illustrates a sample expandable reservoir 106 that may be used in the EFD and reservoir 100 in FIG. 1A or as an excess reservoir in sample configurations. As illustrated, the expandable reservoir 106 may contain one or more connectors or valves 114 on the inlet tube 102 and/or the outlet tube 104. The size of the tubing may be 1-10 mm, while the size of the expandable reservoir 106 is selectable in accordance with the application and may be small (e.g., 1-100 cc) or large (e.g., 100-5000 cc) in different configurations.

Additional configurations of the expandable flow detector and reservoir 100 having additional outlet and/or inlet ports is shown in FIGS. 2A-2D.

For example, FIG. 2A illustrates an example of an EFD and reservoir 200 having two inlet ports 102 and 202 in a sample configuration. The second inlet port 202 may be used, for example, to remove air from the system, to sample the lymphatic fluid, or to add medicine to the lymphatic fluid in the expandable reservoir 204 in sample configurations.

FIG. 2B illustrates a sample expandable reservoir 204 that may be used in the EFD and reservoir 200 in FIG. 2A or as an excess reservoir in sample configurations. As illustrated, the expandable reservoir 204 may contain one or more connectors or valves 114 on the inlet tubes 102 and 202 and/or the outlet tube 104. The size of the expandable reservoir 204 is selectable in accordance with the application and may be small (e.g., 1-100 cc) or large (e.g., 100-5000 cc) in different configurations.

FIG. 2C illustrates another example of an EFD and reservoir 210 having two inlet ports 102 and 202 and two outlet ports 104 and 212 in a sample configuration. The second inlet port 202 may be used, for example, to remove air from the system, to sample the lymphatic fluid, or to add medicine to the lymphatic fluid in the expandable reservoir 214 in sample configurations. The second outlet port 212 may be used, for example, to provide separate outputs to the venous system and a lymph storage system.

FIG. 2D illustrates a sample expandable reservoir 214 that may be used in the EFD and reservoir 210 in in FIG. 2C or as an excess reservoir in sample configurations. As illustrated, the expandable reservoir 214 may contain one or more connectors or valves 114 on the inlet tubes 102 and 202 and/or the outlet tubes 104 and 212. The size of the expandable reservoir 214 is selectable in accordance with the application and may be small (e.g., 1-100 cc) or large (e.g., 100-5000 cc) in different configurations.

In certain configurations, the EFD 100 can include tubular elements 102 and 104 that connect the components of the EFD 100 together, and can also allow the EFD 100 to have a central lumen through which the lymphatic fluid can flow. In certain configurations, the EFD and reservoir 100 is connected to the thoracic duct through a tubular element (also referred to herein as an inflow or inlet tubular member 102), and a component at the distal end is connected to the venous system through a second tubular element (also referred to herein as an outflow or outlet tubular member 104).

In certain configurations, the EFD 100 can have an additional lumen that can connect to the inflow tubular member 102 or the outflow tubular member 104 for delivery of therapeutics or for fluid removal. In certain configurations, a lumen of the device, e.g., a lumen of one or more tubular members 102 or 104 of the device, can collapse in the absence of lymphatic fluid flow. Collapse of the EFD's lumen can prevent blood from flowing from the venous system into the device and/or lymphatic system. For example, and not by way of limitation, the tubular members 102 and 104 of the device can include a collapsing chamber, which can be compressed externally or internally.

In certain configurations, the tubular element 102 inside the thoracic duct and/or the tubular element within the venous system (i.e., the outflow tubular member 104) can have an anchoring element or support structure, such as a balloon-expanding structure, scaffold or self-expanding structure. In such configurations, a tubular element of a configuration of the disclosed device can include a stent that is coupled to its free end as described in U.S. Pat. No. 10,639,458 incorporated by reference herein. For example, and not by way of example, the stent can surround the tubular member 102 or can be adjacent to the tubular member 102. In such configurations, such structures can be used to keep the thoracic duct open and unobstructed and/or can serve to provide a scaffold for the coupled tubular member. The tubular elements 102 and 104 can be perforated to allow better flow of lymph fluid into the element.

Additional configurations of an EFD and reservoir 100 are shown in FIGS. 3-3C. For example, FIG. 3A illustrates an example of an EFD and reservoir 300 including a movable element (e.g., piston) 302 in a sample configuration. Unlike the configuration of FIGS. 1 and 2 that dampens an immediate response using an expandable reservoir, the piston 302 may enable measurement of the fluid in the housing 304 that is displaced by the expandable reservoir 106. The expandable reservoir 106 is designed to operate when partially full and provides resistance to displace the fluid. The displacement may be measured to determine the pressure and/or flow change.

FIG. 3B illustrates another example of an EFD and reservoir 310 including a fixed housing 312 without an expandable reservoir but with a movable piston 314 in a sample configuration. In this example, the lymphatic fluid filling the housing 312 may apply pressure directly against the piston 314 to cause movement of the piston 314. This displacement may be measured to determine the pressure change.

FIG. 3C illustrates another example of an EFD and reservoir 320 including a movable housing element 322 in a sample configuration. In this configuration, rather than a piston, the housing element 322 is movable in response to pressure applied by the fluid within the housing 322 as a result of expansion of the expandable reservoir 106 as the expandable reservoir is filled with lymphatic fluid during use. As illustrated, the housing element 322 moves left and right in response to the applied pressure.

FIG. 4A illustrates an example of an EFD and reservoir 400 surrounded by a fluid 402 such as water, where the water volume may be used to determine the volume of lymphatic fluid stored in the expandable reservoir 106. As illustrated, the EFD and reservoir 400 in this configuration includes and inlet tube 102, an outlet tube 104, and an expandable reservoir 106 made of polyvinyl chloride (PVC), silicone, nylon, rubber, or another suitable expandable material that may expand under pressure and/or flow and deflate when the pressure and/or flow is reduced. In the configuration of FIG. 4A, the expandable reservoir 106 is disposed within a rigid sealed housing 108 that may include a fluid (e.g., water or air) 402 between the housing 108 and the expandable reservoir 106. A fluid chamber 404 may be used to measure changes in volume of the fluid 402 as a result of the pressure applied by the expandable reservoir 106 during use. The sensor 406 may be used to detect changes in fluid level as the expandable reservoir 106 expands or contracts by measuring changes in impedance, resistance, laser contact, etc. The fluid chamber 404 may be connected to a support 408 in an adjustable manner whereby the height of the fluid chamber 404 and/or reservoir 106 relative to the housing 108 may be adjusted.

FIG. 4B illustrates another example of an EFD and reservoir 410 surrounded by a fluid such as water or air, where the water or air volume may be used to determine the volume of lymphatic fluid stored in the expandable reservoir 106. As illustrated, the EFD and reservoir 410 in this configuration includes and inlet tube 102, an outlet tube 104, and an expandable reservoir 106 made of PVC, silicone, nylon, rubber, or another suitable expandable material that may expand under pressure and/or flow and deflate when the pressure and/or flow is reduced. In the configuration of FIG. 4B, the expandable reservoir 106 is disposed within a rigid sealed housing 108 that may include a fluid 402 between the housing 108 and the expandable reservoir 106. A fluid chamber 404 may be used to measure changes in volume of the fluid 402 as a result of the pressure applied by the expandable reservoir 106 during use. The sensor 406 may be used to detect changes in fluid level as the expandable reservoir 106 expands or contracts by measuring changes in impedance, resistance, laser contact, etc. The fluid chamber 404 may be connected to a support 408 in an adjustable manner whereby the height of the fluid chamber 404 relative to the housing 108 may be adjusted. In this configuration, the expandable fluid reservoir 412 is also mounted on the support and connected to the outlet tube 104 to capture lymphatic fluid overflow. Relative heights between the expandable reservoir 106 and expandable fluid reservoir 412 may determine how much of the lymphatic fluid is stored in the expandable fluid reservoir 412.

The expandable flow detectors and reservoirs described herein may be configured in manual, semi-automatic, or automatic pump systems as illustrated in FIGS. 5-7 below.

FIG. 5A illustrates an example of a system 500 for manual or semi-automatic operation of a lymphatic pump 510 where the excess fluid reservoir 520 is located post the EFD and reservoir 530 in a sample configuration. As illustrated, a catheter 540 is inserted into a subject's thoracic duct. The catheter 540 includes a pressure sensor 542 that is located on a distal end of the catheter 540 and that is located within the thoracic duct when the catheter 540 has been inserted into the thoracic duct. A pressure sensor 544 is located on the proximal end of the catheter 540 along with a flow meter 546. The lymphatic fluid extracted from the thoracic duct by the catheter 540 is provided to the EFD and reservoir 530, which may have any of the configurations described above with respect to FIGS. 1-4 and is configured to set a steady state flow. The EFD and reservoir 530 may further include sensors 532 and 534, which may be pressure, flow, temperature, weight, contact, impedance, and/or volume sensors adapted to measure displacement of the EFD and reservoir 530. A pressure sensor 536 also may be provided to track changes in the pressure of the fluid or air as the expandable member 530 expands or contracts. An outlet tube of the EFD and reservoir 530 may be provided to a valve 550 that controls flow of the lymphatic fluid to the expandable or fixed excess fluid reservoir 520, which may also have a sensor 522 and an outlet valve 524. The sensor 522 may measure weight, pressure, a difference in height, and the like. The expandable or fixed excess fluid reservoir 520 provides a low resistance storage that prevents air from being sucked into the pump 510.

Excess lymphatic fluid output by the EFD and reservoir 530 is selectively provided via the valve 550 and filter 552 to the pump 510, which may be a peristaltic pump, for buffering and pumping back into the subject. The pump 510 is powered by an energy source 560, which may be a battery or the output of an AC/DC converter connected to a mains source. The pump 510 returns a percentage of the lymphatic fluid flow back into the venous system of the subject to prevent clotting within the venous system and within the pump 510. Excess fluid may be removed, optionally modified, and stored in the expandable or fixed excess fluid reservoir 520. Prior to being inserted back into the body, the lymphatic fluid output by the pump 510 may be heated to body temperature by a warmer 570, which may be inside or outside the subject. The fluid output of the warmer 570 may be reinserted into the veins of the subject by catheter 580, which may also include a pressure sensor 582 that tracks the pressure of the fluid inserted into the proximal end of the catheter 580. Programming and operation of the elements of FIG. 5A may be provided via computer interface 590 in sample configurations.

FIG. 5B illustrates an example of a system 500′ for manual or semi-automatic operation of a lymphatic pump 510 where the only fluid reservoir 520 is used as the EFD in a sample configuration. In the configuration of FIG. 5B, the EFD and reservoir 530 prior to the valve 550 is used as the expandable or fixed fluid reservoir 520, which may also have a sensor 522 and an outlet valve 524. The operation is otherwise the same as for the system 500 of FIG. 5A.

FIG. 5C illustrates an example of a system 500″ for manual or semi-automatic operation of a lymphatic pump 510 where the fluid reservoir 520 is connected directly to the reservoir of the EFD 530 in a sample configuration. In the configuration of FIG. 5C, an output of the reservoir of the EFD 530 is provided directly to the expandable or fixed fluid reservoir 520, which may also have a sensor 522 and an outlet valve 524. The operation is otherwise the same as for the system 500 of FIG. 5A.

FIG. 6A illustrates an example of a system 600 for automatic operation of a lymphatic pump where the reservoir or outlet 520 is located post the pump 510 in a sample configuration. In the configuration of FIG. 6A, the EFD and reservoir 530 (which may be very small in size) passes lymphatic fluid to the pump 510, which may be an intravenous pump or a peristaltic pump. In this configuration, the pressure sensor 536 may provide a signal to signal processing and filtering element 610 for purposes of determining a variability in the fluid flow rate and to smooth out the fluid flow to avoid drastic movements. The controller 620 receives a processing output of the signal processing and filtering element 610 and controls the pump 510. The output of the pump 510 is provided to the valve 550 and selectively provided to the expandable or fixed fluid reservoir 520 with sensor 522 and outlet valve 524, as appropriate to manage the fluid flow. The remainder of the lymphatic fluid is returned to the subject via warmer 570 and catheter 580 as described above to, for example, maintain fluid levels to avoid dehydration of the subject. The pressure of the lymphatic fluid may be measured at several stages of the process and provided to the controller 620 to control the pump 510 and to optimally manage the fluid flow by, for example, identifying points where the pressure is reduced or significantly increased (and hence indicative of clogging).

In sample configurations, the controller 620 can function to control the one or more components of the device. For example, the controller 620 can function to control the activation, deactivation and/or pressure level of the EFD and reservoir 530. Additionally or alternatively, the controller 620 can control the activation, deactivation and/or level of activity of the pump 510, e.g., flow rate, or the energy source 560 using any combination of a proportional, integral, and/or derivative closed loop pump control with or without a first order filter. In certain configurations, the controller 620 can control the activation, deactivation and/or level of activity of the one or more valves 550, 524, etc. The controller 620 can be located within the subject (i.e., internally) or can be positioned externally, and can communicate with the components of the device, e.g., pump 510, through a wired and/or wireless connection. For example, the controller 620 of the present disclosure can be located external to the body of the subject (see configuration of FIG. 7A).

FIG. 6B illustrates an example of a system 600′ for automatic operation of a lymphatic pump 510 where the reservoir 520 is located post the pump 510 and a second lymphatic pump 630 is provided for partial lymphatic fluid return operation in a sample configuration. In this configuration, the second pump 630 is configured to receive lymphatic fluid from valve 550 that is pumped by second pump 630 to the expandable or fixed excess fluid reservoir 520, with sensor 522 and outlet valve 524. The overflow lymphatic fluid provided to the expandable or fixed excess fluid reservoir 520 may be removed from the system or returned to the subject, for example, via the warmer 570 and the catheter 580 inserted into the subject's vein.

FIG. 6C illustrates an example of a system 600″ for automatic operation of a lymphatic pump 510 where the reservoir or outlet 520 is located before the pump 510 in a sample configuration. As in the configuration of FIG. 5A, in the configuration of FIG. 6C the expandable or fixed fluid reservoir 520 is located before the pump 510 and is configured to receive the lymphatic fluid from an outlet port of the EFD and reservoir 530 as appropriate to maintain a steady state flow of lymphatic fluid to the pump 510.

In the above configurations, the energy source 560 for powering the components of the device can be internal, external and/or integrated into the device. For example, but not by way of limitation, the energy power source 560 can be implanted subcutaneously and coupled to any one of the components of the device, e.g., the pump 510 and controller 620. The energy source 560 can be rechargeable and/or inductive. Any energy source 560 known in the art can be used in the device. Non-limiting examples of energy sources 560 are disclosed in U.S. Patent Application Nos. 2013/0320773 and 2009/0326597 and U.S. Pat. Nos. 8,630,717; 6,640,137; 7,729,768; and 5,810,015, the contents of which are hereby incorporated by reference in their entireties.

In certain configurations, the sensors described herein can be located in the thoracic duct, within the device, e.g., adjacent to the EFD and reservoir 100 and/or pump 510, the venous system, e.g., subclavian vein and/or the innominate vein, at the junction between the thoracic duct and the venous system, at the junction between the device and the thoracic duct and/or at the junction between the device and the venous system. The sensors can include, but are not limited to, pressure sensors, temperature, pH sensors, electrical sensors, piezo sensors, flow sensors, optical sensors, mechanical sensors, force sensors or transducers, blood sensors, infrared sensors, ultrasound sensors and/or volume or fluid level sensors. In certain configurations, the sensors can detect pressure and/or changes in pressure in the thoracic duct, the venous system and/or in the device. In other configurations, the sensors can detect the chemical composition and/or changes in chemical composition of the lymphatic fluid (e.g., acidity, fat content and/or cell composition). The sensors also can detect flow volume and/or changes in flow volume in the thoracic duct, the venous system and/or in the system components, including the EFD and reservoir 100.

In certain configurations, the one or more sensors can function to monitor the pressure of the thoracic duct and be in communication with the controller 620 of the device. In certain configurations, the sensors can signal to the controller 620 to indicate when decompression of the lymphatic system is necessary. For example, the sensors can signal to the controller 620 when the pressure of the thoracic duct is sufficiently high to require flow of the lymphatic fluid into the EFD and reservoir 530. In certain configurations, the detection of a specific indicator, e.g., a specific pressure threshold, can result in the opening of the EFD and reservoir 530 to allow fluid to pass into the EFD and reservoir 530. Alternatively or additionally, the sensors can be coupled to one or more isolation valves, where detection of a specific indicator, e.g., a specific pressure threshold, results in the opening of a valve to allow fluid to pass from the thoracic duct into the EFD and reservoir 530. In other configurations, an isolation valve 550 (FIG. 6B) may function to isolate the system components from the blood of the venous system and/or the lymphatic fluid. In other configurations, the detection of a specific indicator, e.g., a specific pressure threshold, can result in the activation of the pump 510 to pump fluid from the EFD and reservoir 530 to the expandable or fixed fluid reservoir 520 and/or the venous system. Alternatively or additionally, the detection of a specific indicator, e.g., a specific pressure threshold, can result in a change in the rate at which the pump 510 moves fluid from the EFD and reservoir 530 to the expandable or fixed fluid reservoir 520 and/or the venous system, e.g., an increase in the flow rate. The specific pressure threshold at which the device and/or device components can be activated and/or opened depends on the condition of the subject and the type of disease the subject using the device is suffering from.

The one or more sensors can communicate directly with the EFD and reservoir 530, pump 510, and/or valves 550 or indirectly through the controller 620. For example, but not by way of limitation, information from the sensors can be communicated to the controller 620 which can then activate the pump 510. In certain configurations, if the pressure in the thoracic duct is greater than the pressure in the venous system, e.g., subclavian vein and/or the innominate vein, the pump 510 can be activated to remove fluid from the EFD and reservoir 530.

In the above configurations, the device can be implanted within the thoracic duct (TD) or can be positioned at the junction between the TD and the venous system. For example, the device for use in passively decompressing the lymphatic system can be deployed into the TD outlet and extend into the TD and/or the venous system, e.g., subclavian vein and/or the innominate vein. In certain configurations, the valve can be placed inside the TD and/or proximal to the TD/venous junction. In other configurations, the device may be a small wearable or implantable device.

For example, FIG. 7A illustrates an example of a system 700 for automatic operation of a small wearable or implantable device including an EFD and reservoir 530 in a sample configuration. The configuration in FIG. 7A is similar to the configurations described above except that the device components are designed to be small enough to be included in a wearable or implantable device. An isolation valve 710 is also added to provide an outlet to offload excess lymphatic fluid. In addition, a remote or connected computer interface 720 may be provided to communicate with the controller 620 in order to control the operation of the system directly or wirelessly. Alternatively, the computer interface 720 may communicate directly with the other components. The wireless communications may be conducted by BLUETOOTH™ or other standard wireless communications protocols. The computer interface 720 enables the user to program the operation of the pump to maintain desirable thoracic and venous pressures, for example.

In certain configurations, the isolation valve 710 can function to prevent the reflux of blood from the venous system into the TD and/or for preventing the flow of the lymphatic fluid into the EFD and reservoir 530 if decompression of the lymphatic system is not needed. The isolation valve 710 can be of any type known in the art. For example, isolation valve 710 can be a membrane type, a solenoid, a leaflet, a ball, a mechanical, a biological valve or combinations thereof.

FIG. 7B is a plot illustrating the response of a pump to a drop in the EFD pressure set point in a sample configuration. In this example, a drop in EFD pressure at 730 leads to an immediate response by the controller 620 to reduce the pump flow rate at 740. The pump flow rate is reduced until the EFD pressure set point is reset to the set point at 750. The device may then return to steady state operation at the lower pump flow rate.

The internal location, e.g., implantation site, of the device and/or the components of the device can vary depending on the method by which the device/components are to be implanted and/or delivered. For example, and not by way of limitation, the device and/or components of the device can be delivered percutaneously or transcutaneously. In certain configurations, a device of the disclosed subject matter can be placed within the TD. In certain configurations, the device can be placed at the junction between the TD and the venous system, such that part of the device is within the TD and another part of the device is within the venous system, or located within the venous system.

In certain configurations, the components of the disclosed device, e.g., the pump 510 and/or the sensors, can be located within distinct regions of the body of a subject or external to the body of the subject. For example, the pump 510 can be implanted subcutaneously and coupled to an EFD and reservoir 530 that is located within the TD or outside the body. In certain configurations, the inflow tubular member 102 can be positioned within the TD and the outflow tubular member 104 can be placed in the venous system, e.g., within the innominate vein. In certain configurations, the remaining components of the device can be located externally to the TD and/or the venous system. For example, and not by way of limitation, the remaining components of the device can be located external to the body of the subject, e.g., on the surface of the skin of the subject, subcutaneously and/or adjacent to the thoracic duct and/or the venous system. Alternatively, the remaining components of the device can be located within the TD and/or the venous system.

FIGS. 8A and 8B illustrate sample configurations of pumps 800 and 860 for use as the lymphatic pump in sample system configurations. In certain configurations of the disclosed subject matter, the devices described above can include a pump 800 or 860 that can function to actively remove lymphatic fluid from the TD. For example, and not by way of limitation, the pump 800 or 860 can be coupled to an inlet tubular member that is placed within the TD and an outlet tubular member that is positioned with the venous system for pumping lymphatic fluid from the TD into the venous system. The fluid can be pumped from the TD continuously or intermittently. For example, non-limiting examples of pumps that can be used in the system described herein include a piezo pump, a rotary pump, a peristalsis pump, or a reciprocating pump. Non-limiting examples of a rotary pump include a screw, progressing cavity pump, turbine, axial, lobe, centrifuge, and axial rotary pumps. Non-limiting examples of reciprocating pumps include piston, plunger, and diaphragm pumps. Additional non-limiting examples of pumps that can be used in the present disclosure include solenoid pumps, membrane pumps, propellor pumps, as well as pumps of the type described in U.S. Pat. Nos. 8,591,478; 4,969,873; 6,589,198; and 8,034,030; and the like. The contents of these patents are incorporated by reference herein in their entireties.

Peristaltic pumps are positive displacement pumps that insulate the fluid dispensed with the pump mechanism from the external environment. In the proposed configuration of FIG. 8A, the lymphatic fluid passes through a flexible tube 810 within the pump 800. In the configuration of FIG. 8A, the fluid is pushed by a roller 820 moving the fluid forward as a result of a clockwise rotation in the figure. The shoes 830 of the roller 820 pinch the tube 810 and move the fluid in the direction of rotation of the roller 820. The fluid is dispensed from the pump 800 in pulses, and the amount of fluid that is dispensed in one pulse is equal to the volume between the two pinched portions of the tube 810, i.e., the volume of the tube 810 between two shoes 830 of the roller 820. The resolution of the pump 800 is dependent upon the number of shoes 830 and the distance between them on the roller 820. Pressure sensors 840 and 850 may also be provided on the inlet and outlet sides of the pump 800, respectively, to measure any pressure differentials across the pump 800.

FIG. 8B illustrates a linear peristaltic pump configuration in which the fluid within the tube 810 is pushed by linear rollers 870 forward as a result of the linear motion of the rollers 870 towards the exit of the pump 860. As in the configuration of FIG. 8A, the fluid is dispensed from the pump 860 in pulses, and the amount of fluid that is dispensed in one pulse is equal to the volume between the respective rollers 870. Pressure sensors 890 and 890 may also be provided on the inlet and outlet sides of the pump 860, respectively, to measure any pressure differentials across the pump 860.

The selection of the type of pump depends on the location of implantation, condition of the subject using the device and/or the subject's degree of need for assistance in the decompression of the lymphatic system. For example, but not by way of limitation, the pump 800 or 860 can be sufficiently small, e.g., a micropump, to be implanted without the need for major invasive surgery and/or for implantation subcutaneously, within the thoracic duct or the thoracic duct/venous system junction.

In certain configurations, the pump 800 or 860 can pump lymphatic fluid at a rate of about 1 cc/min to about 100 cc/min. The pump 800 or 860 can pump fluid at a variable flow rate or a constant flow rate. The pump 800 or 860 also can be implanted internally, e.g., the pump 800 or 860 can be positioned within the TD. For example, but not by way of limitation, the pump 800 or 860 can be implanted within a stent that is positioned in the TD. Alternatively, the pump 800 or 860 can be positioned within the TD in the absence of a stent. In certain configurations, the pump 800 or 860 can be positioned at the junction between the TD and the venous system. In certain configurations, the pump 800 or 860 can be positioned at the TD/venous system junction within a stent or in the absence of the stent. Alternatively, the pump 800 or 860 can be positioned within the venous system (in the presence or absence of a stent), located external to the body of the subject, e.g., located on the surface of the subject's skin, under the skin (e.g., subcutaneously) or positioned adjacent to the TD and/or innominate vein.

FIG. 9 is a diagram showing the variable placement of the EFD and reservoir 530 in relation to the position of the catheter 540 inserted into the subject. As illustrated, the height H₁ of the expandable flow detector and reservoir 530 may be adjustable over a range of ΔH relative to the position of the catheter 540 inserted into the subject. In this configuration, adjustment of the height H₁ may affect the force of gravity applied to the lymphatic flow.

FIG. 10 is a diagram showing the possibility of the EFD and reservoir 530 at height H₁ and/or the expandable fluid reservoir 520 at height H₂ being adjustable relative to one another in a sample configuration. As illustrated, the height H₁ of the EFD and reservoir 530 relative to the height H₂ of the expandable fluid reservoir 520 may be adjustable over a range of ΔH. In this configuration, adjustment of the relative height ΔH may affect the force of gravity applied to the lymphatic flow.

In either of the configurations of FIG. 9 or FIG. 10, the reservoir height may be changed to change resistance. The change in height may change where the fluid from the EFD and reservoir 530 goes and may change TD outlet pressure.

Any or all components of the devices described herein can be made from, for example, single or multiple stainless steel alloys, nickel titanium alloys, cobalt-chrome alloys, nickel-cobalt alloys, molybdenum alloys, tungsten-rhenium alloys, polymers such as polyethylene terephthalate (PET), polyester, polyester amide, polypropylene, aromatic polyesters, such as liquid crystal polymers, ultra-high molecular weight polyethylene fiber and/or yarn, polytetrafluoroethylene (PTFE), expanded PTFE (ePTFE), polyether ketone (PEK), polyether ether ketone (PEEK), polyether ketone (PEKK), nylon, polyether-block co-polyamide polymers, aliphatic polyether polyurethanes, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials or combinations thereof. Non-limiting examples of radiopaque materials are barium sulfate, zinc oxide, titanium, stainless steel, nickel-titanium alloys, tantalum, and gold. Alternatively or additionally, one or more components of the disclosed devices can be made from a biomaterial or coated with a biomaterial. Non-limiting examples of a biomaterial include tissue, collagen, allograft, autograft, heterograft, xenograft, bone cement, morselized bone, osteogenic powder and beads of bone. Alternatively or additionally, one or more components of the disclosed devices can be coated with a polymer, chemical or biomaterial to make it more biocompatible or hydrophilic, or to prevent deposition of lipids or protein onto the walls and/or surfaces of the device. Also, the catheters, tubing, and pump elements may be coated with an anticoagulant such as heparin.

In certain configurations, the device can further include one or more ports that can be accessed intermittently or continuously. The port can be located at any location on the device. For example, and not by way of limitation, the port can be accessed externally by the presence of a catheter (or tubular member) that is coupled to the port. Alternatively or additionally, the port can be accessed percutaneously. Non-limiting examples of ports that can be present on the device include self-healing ports. In certain configurations, the port can be used to introduce therapeutics into the device and/or the venous system of the subject (e.g., in the case of an occlusion). Non-limiting examples of therapeutics that can be introduced through a port present within the device include anti-coagulants such as factor Xa inhibitors, heparin and tissue plasminogen activator (tPA). In certain configurations, the port can be used to flush the device and/or the one or more tubular members of the device.

In certain configurations, the device can further include a component that is located proximally to the pump 510 for preventing the accumulation of protein within the pump 510. For example, and not by way of limitation, the device can include rotating blades that can function to break down protein that is present in the lymph fluid prior to entrance of the fluid into the pump 510.

### Methods of Use

The presently disclosed subject matter further relates to methods for alleviating the central lymphatic congestion using one or more of the devices disclosed above. For example, but not by way of limitation, the present disclosure provides methods for delivery of the disclosed devices within the TD.

In certain configurations, the implantation of a device of the present disclosure can be accomplished using minimally invasive or surgical techniques. By using minimally invasive techniques, the complications associated with open surgery, e.g., higher risks of infection and longer recovery times, can be avoided. For example, a device disclosed herein can be inserted into the venous system, e.g., subclavian vein, percutaneously. In certain configurations, the device can be inserted into the venous system and guided through the venous system to access the TD. For example, the method can include the use of a delivery catheter and/or a tubular member to deliver the device to the TD and/or thoracic duct outlet. In certain configurations, the tubular members 102 and 104 may include outlets (or ports) positioned near the distal end of the member to allow fluid drainage after implantation of the device.

In certain configurations, a device disclosed herein can be inserted into the thoracic directly, without entering the venous system. For example, and not by way of limitation, the device can be inserted into the TD surgically, transcutaneously or percutaneously. In certain configurations, the device can be inserted into the TD and guided through the TD for proper placement. The method can include the use of a wire, delivery catheter and/or a tubular member to deliver the device to the TD and/or TD outlet. In certain configuration, the wire, delivery catheter and/or a tubular member can be inserted into the TD retrograde or anterograde and can be used to guide the device to its proper location. In certain configurations, one or more components of the device can be inserted into the TD directly and one or more components of the device can be inserted into the venous system, e.g., innominate vein, directly.

In certain configurations, the tubular member used for delivering the device is tapered at its distal end, for example, to a thin wire at its distal end, where the wire can assist in guiding the tubular member through the venous system. For example, a device that includes a self-expanding stent can be deployed by advancing the stent out of an end of a tubular member, e.g., a delivery catheter, at the site of implantation. In certain configurations, the method can include the use of a retractable sheath coupled to the tubular member to implant the device. In certain configurations, a device can be positioned inside a tubular member and internally implanted by unsheathing of an outer sheath from the tubular member. For example, a device that includes a stent can be deployed by the retraction of an outer sheath from a tubular member that restrained the self-expanding stent in its collapsed state. Alternatively or additionally, a device can be positioned at the tip of a tubular member and delivered to the site of implantation, e.g., the junction between the TD and the venous system.

In certain configurations, the method can include dilating the TD outlet prior to implantation of the device. The TD outlet can be dilated through the use of a stent, a balloon, a dilator and other expanding devices. For example, but not by way of limitation, the method for delivery of a device disclosed herein can include the use of a tubular member that includes a balloon and/or dilator to dilate the TD outlet, and deliver the device to the site of dilation. In certain configurations, the dilator is tapered at its distal end.

In certain configurations, the method can include the separate delivery of one or more components of a device to the site of implantation followed by the assembly of the device at the site of implantation. For example, the method can include the delivery of the EFD and reservoir 530 followed by the delivery of the pump 510, where coupling of these two components to generate an integrated device can occur at the site of implantation.

In certain configurations, the devices of the disclosed subject matter can be disposable and deployed within a stent or a tubular element and can be easily retrieved and replaced. In certain configurations, the device can have an open pathway that allows for passive decompression and, in addition, can have an active pathway that passes through the EFD and reservoir 530 and the pump 510 for additional active decompression when needed.

FIG. 11A is a diagram showing possible catheter and cannula positions and a TD entrance in a sample configuration. In this example, a catheter or cannula 1100 is inserted near the clavicle of the subject and guided to the TD or the subclavian vein. On the other hand, as shown in FIG. 11B, the catheter or cannula 1100 may be inserted transcervically and guided to the TD 1110 adjacent the innominate vein 1120. As another alternative, FIG. 11C is a diagram showing a venous approach via an internal jugular vein (not shown), innominate vein 1120, subclavian vein 1130, or any other vein suitable for inserting the catheter and cannula 1100 into the TD 1110. FIG. 11D is a diagram showing a surgical approach to inserting the catheter and cannula into the TD 1110 in a sample configuration. In this surgical example, the TD 1110 is accessed surgically and a slit is cut into the TD 1110 for attachment (e.g., by sutures) of a tube 1140 to the wall of the TD 1110. The tube 1140 may be a stented graft on the distal end and include a connector at the surface of the skin on the proximal end. The tube 1140 may be made of any suitable material including GORE-TEX®, Dacron, polyester, plastic, polymers, Nitinol, and the like. In any of these examples, the catheter or cannula 1100 may be inserted into the subject's body and left in place for a period of time as needed to drain the lymphatic fluid continuously or intermittently from the subject. Thus, like dialysis, the catheter or cannula 1100 may be left in place for periodic treatments.

FIG. 12 illustrates an example TD cannula and catheter 1100 having a portion 1200 of the catheter that is more flexible and braded to allow bending but preventing compression in a sample configuration. The portion 1200 may also be self-expanding. As illustrated, the catheter or cannula 1100 is shaped and includes another portion 1210 that can be made of the same or different material than the portion 1200. The respective portions 1200 and 1210 may be separated by a bend 1220. The cannula or catheter 1100 further includes a connector or access port 1230 and a low profile anchor or cuff 1240.

FIGS. 13A-13P illustrate respective alternative configurations of a TD cannula and catheter 1100 adapted for inserting into the TD. Each TD cannula and catheter 1100 is designed from a suitable material so as to be flexible but not collapsible.

The catheter or cannula configuration of FIG. 13A is characterized by holes 1300 that provide fluid access to the internal lumen of the catheter or cannula.

The catheter or cannula configuration of FIG. 13B is characterized by a balloon 1310 that is configured to occlude the TD outlet upon insertion into the TD to hold the catheter or cannula in place and to prevent blood from refluxing back into the TD upon insertion.

The catheter or cannula configuration of FIG. 13C is similar to that in FIG. 13B except that the balloon 1310 is replaced by an expandable element 1320 such as a foam designed to occlude the TD outlet upon insertion into the TD.

The catheter or cannula configuration of FIG. 13D is characterized by a disk-shaped cuff 1340 adapted to hold the catheter or cannula in place once inserted into the TD.

The catheter or cannula configuration of FIG. 13E is similar to that in FIG. 13D except that two disk-shaped cuffs 1340 are provided to hold the catheter or cannula in place once inserted into the TD.

The catheter or cannula configuration of FIG. 13F is characterized by sensors 1350 disposed at the distal and proximal ends of the catheter or cannula.

The catheter or cannula configuration of FIG. 13G is characterized by inclusion of a dilator tapper 1360 of the type shown, for example, in FIG. 13H.

The catheter or cannula configuration of FIG. 13I is characterized by inclusion of an occluder 1370 of the type shown, for example, in FIG. 13J. The occluder 1370 is provided for when the catheter or cannula is not in use.

The catheter or cannula configuration of FIG. 13K is characterized by an exchangeable catheter designed to fit in the TD sheath 1380.

The catheter or cannula configuration of FIG. 13L is characterized by an anchor 1390.

The catheter or cannula configuration of FIG. 13M is similar to that of FIG. 13A except that it does not include holes 1300.

FIG. 13N illustrates a sample configuration of an occluder 1370 connected to the connector or access port 1230.

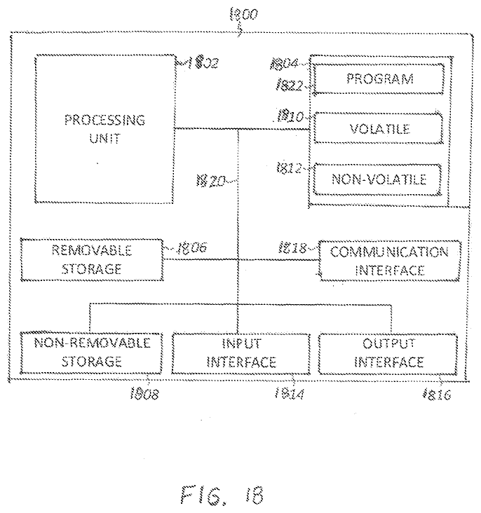

FIGS. 130 and 13P illustrate sample configurations of a catheter or cannula cleaner 1395.

FIGS. 14A-14D illustrate insertion of a TD cannula and catheter into the TD and different mechanisms for holding the TD cannula and catheter in place in sample configurations.

In FIG. 14A, the cannula or catheter of FIG. 13B is inserted into the TD such that the balloon 1310 occludes the TD outlet. Similarly, in FIG. 14B, the cannula or catheter of FIG. 13C is inserted into the TD such that the expandable element 1320 occludes the TD outlet.

In FIG. 14C, the cannula or catheter of FIG. 13E is inserted into the TD such that the TD outlet is disposed between the respective disk-shaped cuffs 1340 to prevent blood from refluxing back into the TD. Similarly, in FIG. 14D, the cannula or catheter of FIG. 13D is inserted into the TD such that the disk-shaped cuff 1340 prevents blood from refluxing back into the TD.

FIGS. 15A-15C illustrate respective catheter configurations for providing side access into the TD in sample configurations.

FIG. 15A illustrates the placement of the TD cannula and catheter of FIG. 13M into the TD 1110. No cuff is provided in this configuration. On the other hand, FIG. 15B illustrates the placement of the TD cannula and catheter or FIG. 13D into the TD 1110 where the disk-shaped cuff 1340 occludes fluid leakage from the TD 1110 when inserted. FIG. 15C illustrates the placement of the TD cannula and catheter 1100 of FIG. 12 into the TD 1110.

As noted above, the purpose of the devices described herein is to provide a lymphatic assist device that decompresses the thoracic duct to minimize accumulation of lymphatic fluid in the subject's tissue by controlling the thoracic duct pressure and lymph flow. In particular, the goal is to control the thoracic duct pressure to remain between 0-40 mm Hg and to decompress the thoracic duct and control the lymph flow out of the thoracic duct. The thoracic duct pressure may be set and controlled to be a fixed difference from the venous pressure (e.g., 4 mm Hg below the venous pressure) in sample configurations. Measurement errors due to respiration, movement, and physiological variations such as heart beat may be removed using signal processing and filtering.

Different modes of operation are contemplated. In manual mode, the operator calculates the flow and programs the pump accordingly. In semi-automatic mode, the flow is calculated automatically and the operator programs the pump based on the calculation. In automatic mode, the calculated flow is automatically used to control the pump operation. In one configuration, the device may be a wearable miniature device for continuous use. In another configuration, the device may be used in a manner similar to a dialysis machine where the subject uses the device as an outpatient and connects to the device for several hours to pump the lymph. In any configuration, the subject may connect to a bag and drain some of the lymph fluid. The connection may last a few minutes or may last for weeks or months in or out of the hospital as needed. All or part of the collected fluid may be returned to the subject or discarded, as appropriate. The device may also be provided in the subject's home for intermittent use.

Flow changes may be detected by detecting changes in pressure, volume, or weight of the lymph fluid. The pressure and/or flow of the lymphatic fluid may be changed by controlling the pressure in the reservoir housing use fluid or gas, by changing the height of the reservoir, by changing the height of fluid in an external chamber connected to the reservoir housing, changing the size of the reservoir housing, and/or by using a piston or other volume changing component in the reservoir housing.

FIG. 16 illustrates the modes of operation of the system of FIG. 15C in a sample configuration. In the example configuration, at least six different pressure sensors (P_{TD}, P₀, P₁, P₂, P₃, and Pᵥ) are provided for management of at least 8 different modes of operation. Measurements by these sensors may be used to identify points of occlusion within the system during trouble shooting.

The sensor P_{TD} is inserted into the TD to determine the fluid pressure within the TD during operation. The sensor P0 is provided on a proximal end of the catheter 540 to determine the pressure at the distal end of the catheter 540. Sensor P₁ determines the pressure within expandable flow detector and reservoir 530. In a first mode of operation, an increase in P₁ represents an increase in pump flow and hence an increased fluid flow in the TD, while in a second mode of operation a decrease in P₁ represents a decrease in pump flow and hence a decreased fluid flow in the TD.

During operation in a third mode, if the pressures measured at sensors P₀, P₁, P₂, P₃, and Pᵥ all decrease, this is indicative of an occlusion in the TD catheter 540, and the pump 510 is stopped.

During operation in a fourth mode, if the pressures measured at sensor P₀ increases but the pressures at P₁, P₂, P₃, and Pᵥ all decrease, this is indicative of an occlusion after the TD catheter 540 but before the EFD and reservoir 530, and the pump 510 is stopped.

During operation in a fifth mode, if the pressures measured at sensors P₀ and P₁ increase but the pressures at P₂, P₃, and Pᵥ all decrease, this is indicative of an occlusion after the EFD and reservoir 530, and the pump 510 is stopped.

During operation in a sixth mode, if the pressures measured at sensors P₀, P₁ and P₂ increase but the pressures at P₃, and Pᵥ decrease, this is indicative of an occlusion in the pump 510, and the pump 510 is stopped.

During operation in a seventh mode, if the pressures measured at sensors P₀, P₁, P₂ and P₃ increase but the pressure at Pᵥ decreases, this is indicative of an occlusion after the pump 510, and the pump 510 is stopped.

During operation in an eighth mode, if the pressures measured at sensors P₀, P₁, P₂, P₃, and Pᵥ all increase, this is indicative of an occlusion in the venous catheter 580, and the pump 510 is stopped.

Thus, the measured pressures may be used to identify the location of an occlusion, which is used to identify the area for unclogging during troubleshooting. For example, the catheter or cannula cleaner 1395 of FIG. 13O or 13P may be inserted into the relevant portion of the tubing to clear any obstruction during troubleshooting.

FIG. 17 illustrates a system 1700 for providing TD outlet pressure control with an automatic pump in a sample configuration. System 1700 is similar to the system 700 of FIG. 7A except that the signal processing and filtering 610 is optional and a set point input 1710 is provided via a dial or a graphical user interface for controlling the set point of the pump 510 and pressure sensor 536 via the controller 620. The system 1700 of FIG. 17 thus provides automatic operation of a device including an expandable flow detector and reservoir 530 as described herein. The configuration in FIG. 17 is similar to the configurations described above except that the device components are designed to be small enough to be included in a wearable or implantable device. The dial or graphical user interface 1710 enables the user to program the operation of the pump 510 to maintain desirable thoracic and venous pressures, for example.

The system 1700 may be operated in several modes. For example, in a normal operating mode, the pressure P₂ of sensor 536 may be set to X₁ by the set point input 1710, where X₁ is the initial set point (e.g., 0 mm Hg). The set point may be any desired outside pressure. In a mode where P₂ is to be increased, the set point is increased and the speed of the pump 510 is decreased until a new set point is reached. The controller 620 then maintains the new set point. On the other hand, in a mode where P₂ is to be decreased, the set point is decreased and the speed of the pump 510 is increased until a new set point is reached. The controller 620 then maintains the new set point. Since the system is closed, the pressure may be calibrated and set to run until the system reaches the set point.

FIG. 18 is a block diagram illustrating circuitry for performing methods and implementing processing features according to example configurations. For example, the processing circuitry of FIG. 18 may be used to implement the controller, the signal processing circuitry, the graphical computer interfaces, and any other processing components described herein. All components need not be used in various configurations.

FIG. 18 illustrates one example of a computing device in the form of a computer 1800 that may include a processing unit 1802, memory 1804, removable storage 1806, and non-removable storage 1808. Although the example computing device is illustrated and described as computer 1800, the computing device may be in different forms in different configurations. For example, the computing device may instead be a smartphone, a tablet, smartwatch, or other computing device including the same or similar elements as illustrated and described with regard to FIG. 18. Devices, such as smartphones, tablets, and smartwatches, are generally collectively referred to as mobile devices or user equipment. Further, although the various data storage elements are illustrated as part of the computer 1800, the storage may also or alternatively include cloud-based storage accessible via a network, such as the Internet or server-based storage.

Memory 1804 may include volatile memory 1810 and non-volatile memory 1812. Computer 1800 also may include, or have access to a computing environment that includes, a variety of computer-readable media, such as volatile memory 1810 and non-volatile memory 1812, removable storage 1806 and non-removable storage 1808. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1800 may further include or have access to a computing environment that includes input interface 1814, output interface 1816, and a communication interface 1818. Output interface 1816 may include a display device, such as a touchscreen, that also may serve as an input device. The input interface 1814 may include one or more of a touchscreen, touchpad, mouse, keyboard, camera, one or more device-specific buttons, one or more sensors integrated within or coupled via wired or wireless data connections to the computer 1800, and other input devices.

The computer 1800 may operate in a networked environment using communication interface 1818 to connect to one or more remote computers. The remote computer may include a personal computer (PC), server, router, network PC, a peer device or other common network switch, or the like. The communication connection accessed via communication interface 1818 may include a Local Area Network (LAN), a Wide Area Network (WAN), cellular, Wi-Fi, Bluetooth, Zigbee, or other networks. According to one configuration, the various components of computer 1800 are connected with a system bus 1820.

Computer-readable instructions stored on a computer-readable medium are executable by the processing unit 1802 of the computer 1800, such as a program 1822. The program 1822 in some configurations comprises software that, when executed by the processing unit 1802, performs operations according to any of the configurations included herein. A hard drive, CD-ROM, and RAM are some examples of articles including a non-transitory computer-readable medium, such as a storage device. The terms computer-readable medium and storage device do not include carrier waves to the extent carrier waves are deemed too transitory. Storage can also include networked storage, such as a storage area network (SAN). Computer program 1822 may be used to cause processing unit 1802 to perform one or more methods or functions described herein.

It should be further understood that software including one or more computer-executable instructions that facilitate processing and operations as described above with reference to any one or all of steps of the disclosure may be installed in and sold with one or more of the systems described herein. Alternatively, the software may be obtained and loaded into the systems in a manner consistent with the disclosure, including obtaining the software through a physical medium or distribution system, including, for example, from a server owned by the software creator or from a server not owned but used by the software creator. The software may be stored on a server for distribution over the Internet, for example.

Also, it will be understood by one skilled in the art that this disclosure is not limited in its application to the details of construction and the arrangement of components set forth in the above description or illustrated in the drawings. The configurations herein are capable of other configurations, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The components of the illustrative devices, systems and methods employed in accordance with the illustrated configurations may be implemented, at least in part, in digital electronic circuitry, analog electronic circuitry, or in computer hardware, firmware, software, or in combinations of them. These components also may be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program may be written in any form of programming language, including compiled or interpreted languages, and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. Also, functional programs, codes, and code segments for accomplishing the systems and methods described herein may be easily construed as within the scope of the disclosure by programmers skilled in the art to which the present disclosure pertains. Method steps associated with the illustrative configurations may be performed by one or more programmable processors executing a computer program, code or instructions to perform functions (e.g., by operating on input data and generating an output). Method steps may also be performed by, and apparatus may be implemented as, special purpose logic circuitry, e.g., an FPGA (field programmable gate array) or an ASIC, for example.

The various illustrative logical blocks, modules, and circuits described in connection with the configurations disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random-access memory or both. The elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, compact disc ROM (CD-ROM), or digital versatile disc ROM (DVD-ROM). The processor and the memory may be supplemented by, or incorporated in, special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those skilled in the art may further appreciate that the various illustrative logical blocks, modules, circuits, and algorithm steps described in connection with the configurations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. A sample storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., EEPROM), and any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, which is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

Although the presently disclosed subject matter and its advantages have been described in detail, it should be understood that various changes, substitutions and alterations can be made herein without departing from the spirit and scope of the presently disclosed subject matter as defined by the appended claims. Moreover, the scope of the presently disclosed subject matter is not intended to be limited to the particular configurations described in the specification. Accordingly, the appended claims are intended to include within their scope such modifications. Various references are cited herein, the contents of which are hereby incorporated by reference in their entireties.

1.-29. (canceled)

30. An expandable flow detector and reservoir adapted to measure flow of lymphatic fluid in a device for reducing congestion of a lymphatic system, comprising:
an inlet tubular member that receives lymphatic fluid from the lymphatic system;
an expandable reservoir that expands when at least one of pressure or flow increases and deflates when the at least one of pressure or flow decreases;
a sealed housing around the expandable reservoir including a fluid that is compressed as the reservoir expands;
an outlet tubular member that provides the lymphatic fluid to a pump; and
a sensor that measures a change in at least one of volume or pressure of the expandable reservoir as the expandable reservoir expands or contracts in response to flow of the lymphatic fluid.

31. The expandable flow detector and reservoir of claim 30, wherein the expandable reservoir comprises at least one connector or valve that receives the inlet tubular member and at least one connector or valve that receives the outlet tubular member.

32. The expandable flow detector and reservoir of claim 30, further comprising a movable element that is displaced as the expandable reservoir expands, whereby a displacement of the movable element is indicative of a change in at least one of pressure or flow in at least one of the expandable reservoir or the sealed housing.

33. The expandable flow detector and reservoir of claim 30, wherein the expandable reservoir comprises the sealed housing and a movable piston that is displaced as lymphatic fluid is received from the lymphatic system, whereby a displacement of the movable piston is indicative of a change in an amount of received lymphatic fluid.

34. The expandable flow detector and reservoir of claim 30, wherein the sealed housing comprises a movable housing element that is movable in response to at least one of pressure or flow as a result of the volume of fluid within the sealed housing expanding as the expandable reservoir is filled with lymphatic fluid.

35. The expandable flow detector and reservoir of claim 30, further comprising a fluid chamber that communicates with the fluid in the sealed housing, wherein the sensor is adapted to measure changes in volume of the fluid in the fluid chamber as a result of changes in at least one of pressure or flow of the lymphatic fluid received by the expandable reservoir as the expandable reservoir expands or contracts during use.

36. The expandable flow detector and reservoir of claim 35, further comprising a support for the fluid chamber, wherein the fluid chamber is connected to the support in an adjustable manner whereby a height of the fluid chamber relative to the sealed housing may be adjusted.

37. The expandable flow detector and reservoir of claim 36, further comprising an expandable excess fluid reservoir that captures fluid overflow based on a relative level of the expandable excess fluid reservoir to the fluid chamber.

38. A device for reducing congestion of a lymphatic system, comprising:
a first catheter or cannula adapted for insertion into a thoracic duct of a subject to extract lymphatic fluid from the thoracic duct of the subject;
an inlet tubular member that receives lymphatic fluid from the first catheter or cannula;
an expandable reservoir that receives the lymphatic fluid from the inlet tubular member and expands when at least one of pressure or flow increases and deflates when the at least one of pressure or flow decreases;
a sealed housing around the expandable reservoir including a fluid that is compressed as the expandable reservoir expands;
a sensor that measures a change in at least one of volume or pressure of the expandable reservoir as the expandable reservoir expands or contracts in response to flow of the lymphatic fluid.
a pump;
an outlet tubular member that outputs the lymphatic fluid from the expandable reservoir to the pump; and
a second catheter or cannula that receives the lymphatic fluid from the pump and is adapted for insertion into a vein of a subject to insert lymphatic fluid into the subject.

39. The device of claim 38, further comprising an expandable or fixed excess fluid reservoir connected between the expandable reservoir and the pump and adapted to receive excess lymphatic fluid.

40. The device of claim 38, further comprising an expandable or fixed excess fluid reservoir connected directly to the expandable reservoir within the sealed housing and adapted to receive excess lymphatic fluid.

41. The device of claim 38, further comprising an expandable or fixed excess fluid reservoir connected after the pump and adapted to receive excess lymphatic fluid.

42. The device of claim 38, further comprising a signal processor and filter that receives a signal from the sensor and determines a variability in a fluid flow rate through the expandable reservoir and filters out noise signals.

43. The device of claim 42, further comprising a controller that receives a processing output from the signal processor that is used to control the pump, wherein the controller controls at least one of activation, deactivation or pressure level of at least one of the expandable reservoir or the pump, and wherein the controller is positioned remotely from the pump and communicates with the pump through at least one of a wired or wireless connection.

44. The device of claim 43, further comprising a second pump that pumps excess lymphatic fluid output by the pump to an expandable or fixed excess fluid reservoir.

45. The device of claim 43, further comprising an expandable or fixed excess fluid reservoir connected to an output of the expandable reservoir.

46. The device of claim 43, further comprising an input device that provides a set point to the controller for controlling the pump and the sensor to maintain desired thoracic duct outlet and interstitial pressures.

47. The device of claim 38, wherein the expandable reservoir, the sealed housing, the sensor, and the pump have sizes adapted to enable the device to be insertable into a thoracic duct, a subclavian or other vein of a subject surgically or transcutaneously, inserted subcutaneously or into a compartment of the subject, or into a wearable.

48. The device of claim 38, wherein the pump is controlled to increase pump flow when the sensor detects increased lymphatic fluid flow or pressure and to decrease pump flow when the sensor detects decreased lymphatic fluid flow or pressure.

49. The device of claim 38, further comprising a plurality of pressure sensors placed at one or more locations including a proximal end of the first catheter or cannula, a proximal end of the second catheter or cannula, at an entrance to the pump, and an exit of the pump, wherein a location of an occlusion in the device is detected by detecting a pressure change between respective pressure sensors.
